(19) United States
(12) Patent Application Publication
(10) Pub. No.: US 2026/0077043 A1
(43) Pub. Date: Mar. 19, 2026

(54) COMBINATION THERAPY

(71) Applicant: LEAP THERAPEUTICS, INC., Cambridge, MA (US)

(72) Inventors: Michael H. Kagey, Arlington, MA (US); Cynthia A. Sirard, Cambridge, MA (US); Michael Haas, Chelmsford, MA (US)

(21) Appl. No.: 18/993,300

(22) PCT Filed: Jul. 12, 2023
(86) PCT No.: PCT/US2023/027524

§ 371 Date: Jan. 10, 2025

Related U.S. Application Data
(60) Provisional application No. 63/388,553, filed on Jul. 12, 2022.

(51) Int. Cl. A61K 39/395; A61K 31/282; A61K 31/4439; A61K 31/513; A61K 31/519; A61K 39/00; A61P 35/00

(52) U.S. Cl. CPC A61K 39/39558; A61K 31/282; A61K 31/4439; A61K 31/513; A61K 31/519; A61P 35/00; A61K 2039/507; A61K 2039/545

(57) ABSTRACT

The invention relates to methods of treating colorectal cancer in a subject in need thereof. The methods comprising co-administering to the subject: a) a DKK1 antibody, or antigen binding-fragment thereof; b) a VEGF or VEGFR inhibitor; and c) optionally one or more chemotherapeutic agents or a pharmaceutically acceptable salt of any of the foregoing, in an effective amount. The present invention also provides a pharmaceutical composition and a kit comprising a combination of a DKK1 antibody, and a VEGF or VEGFR inhibitor and optionally one or more chemotherapeutic agents.

### RELATED APPLICATION

This application claims the benefit of U.S. Provisional Application No. 63/388,553, filed on Jul. 12, 2022. The entire teachings of the above application are incorporated herein by reference.

### BACKGROUND OF THE INVENTION

Colorectal cancer (CRC) is the third most prevalent cancer, with 1,931,590 cases diagnosed in 2020, and the second leading cause of cancer-related death, with 935,173 deaths in 2020 worldwide (World Health Organization 2021). The global prevalence of CRC is higher in men than in women (746,298 versus 614,304) (White et al, 2018). Despite the favorable contribution of screening programs, 25% of patients have advanced disease at diagnosis, whereas an additional 50% subsequently advance to metastases, accounting for a higher mortality rate (Van Cutsem et al, 2011). Advanced colorectal cancer (CRC) continues to be a life-threatening and serious disease. As such, there is a continuing need for new and improved treatments for patients with cancer.

### SUMMARY OF THE INVENTION

The invention described herein relates to a method of treating colorectal cancer in a subject in need of treatment.

In a first embodiment, the method comprises co-administering to the subject suffering from colorectal cancer a DKK1 antibody, or antigen binding-fragment thereof, a VEGF or VEGFR inhibitor; and optionally one or more chemotherapeutic agents or a pharmaceutically acceptable salt of any of the foregoing, in an effective amount.

In another embodiment, the invention relates to a pharmaceutical composition comprising: a DKK1 antibody, or antigen binding-fragment thereof; bevacizumab; and optionally one or more chemotherapeutic agents or a pharmaceutically acceptable salt of any of the foregoing.

In yet another embodiment, the present invention is a pharmaceutical composition comprising: a) a DKK1 antibody, or antigen binding-fragment thereof; b) a VEGF or a VEGFR inhibitor; and c) one or more chemotherapeutic agents or a pharmaceutically acceptable salt of any of the foregoing.

In yet another embodiment, the present invention is a pharmaceutical composition comprising: a) a DKK1 antibody, or antigen binding-fragment thereof; b) a VEGF or a VEGFR inhibitor; and c) one or more chemotherapeutic agents for use in the treatment of colorectal cancer.

In yet another embodiment, the present invention is a pharmaceutical composition comprising: a) a DKK1 antibody, or antigen binding-fragment thereof; b) a VEGF or a VEGFR inhibitor; and c) one or more chemotherapeutic agents for use in the preparation of a medicament for the treatment of colorectal cancer.

In yet another embodiment, the present invention is a kit comprising: a) a DKK1 antibody, or antigen binding-fragment thereof; b) a VEGF or a VEGFR inhibitor; c) one or more chemotherapeutic agents or a pharmaceutically acceptable salt thereof; and d) instructions for use.

In yet another embodiment, the present invention is a kit comprising: a) a DKK1 antibody, or antigen binding-fragment thereof; b) bevacizumab; c) one or more chemotherapeutic agents or a pharmaceutically acceptable salt thereof; and d) instructions for use.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 depicts the overall design of a randomized phase 2 clinical trial.

FIGS. 2A and 2B shows the results of co-administration of DKN-01 and the PI3K inhibitor, apelisib, in a xenograft model of colorectal cancer.

FIG. 3 shows the results of duration of co-administration of mDKN-01 and an anti-PD-1 antibody in a xenograft model of colorectal cancer.

FIG. 4 shows a consort diagram of Part A of the DeFianCE study.

FIG. 5 shows Table 1 that characterizes the patients studied in Part A of the DeFianCe study.

FIG. 6 is a plot showing the Best Overall Response of the evaluable patients studied in Part A of the DeFianCe study.

FIG. 7A is a plot showing the Best Overall Response of the evaluable patients studied in Part A of the DeFianCe study, categorized by the presence of KRAS mutations.

FIG. 7B is a plot showing the Best Overall Response of the evaluable patients studied in Part A of the DeFianCe study, categorized by the presence of liver metastases.

FIG. 7C is a plot showing the Best Overall Response of the evaluable patients studied in Part A of the DeFianCe study, categorized by the prior Bevacizumab treatment.

FIG. 7D is a plot showing the Best Overall Response of the evaluable patients studied in Part A of the DeFianCe study, categorized by the rapid progression of disease.

FIG. 8 is a “swimmer” plot showing duration on study, follow-up for overall survival post-therapy and the Best Overall Responseof study to datepatients.

### DETAILED DESCRIPTION OF THE INVENTION

A description of example embodiments of the invention follows.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### Colorectal Cancer

Colorectal cancer (CRC), also known as bowel cancer, colon cancer, or rectal cancer, is any cancer that affects the colon, the rectum or both. The American Cancer Society estimates that about 1 in 21 men and 1 in 23 women in the United States will develop colorectal cancer during their lifetime.

The colorectal cancer can be Stage 0, Stage I, Stage IIA, Stage IIB, Stage IIC, Stage IIIIA, Stage IIIB, Stage IIIC, Stage IVA, Stage IVB or Stage IVC as defined by the TNM system of the American Joint Committee on Cancer (AJCC).

In some embodiments, the colorectal cancer is metastatic or advanced colorectal cancer (i.e., colorectal cancer that has spread to other parts of the body from the primary site such as to the lungs, liver or any other organ other than the primary site). The methods described herein are directed to the treatment of a subject with colorectal cancer. In some embodiments, the colorectal cancer is colorectal adenocarcinoma. In some embodiments, the colorectal cancer is metastatic colorectal cancer (mCRC). In some embodiments, the colorectal cancer is proficient mismatch repair (pMMR) mCRC. In some embodiments, the CRC is not pMMR. In some embodiments, the CRC is deficient mismatch repair (dMMR). In some embodiments, the colorectal cancer is microsatellite stable (MSS) CRC. In some embodiments, the colorectal cancer is microsatellite instable (MSI) CRC. In some embodiments, the colorectal cancer is pMMR/MSS mCRC. In some embodiments, the colorectal cancer is pMMR/MSS mCRC and has not been responsive to previous treatment with single agent checkpoint inhibitors. In some embodiments the CRC is KRAS wild type and has not been responsive to previous treatment with EGFR inhibitors. In some embodiments, the colorectal cancer is an advanced cancer. In some embodiment, the colorectal cancer is specific to the rectum. In another embodiment, the colorectal cancer is specific to the colon. In yet another embodiment, the colorectal cancer involves both the colon and the rectum.

MSS CRC: Each of our cells contains DNA with genes that provide instructions for our cells on how to grow, carry out specific activities, divide, or die. The DNA in our cells also contains segments of short repetitive DNA sequences called microsatellites. This microsatellite DNA serves as a biomarker for how stable our DNA is. The DNA is considered stable when the number of microsatellite repeats is the same in all the cells of the body, also referred to as microsatellite stable or MSS.

DNA mismatch repair (MMR) is a quality control and “spell checking” process that is responsible for making sure the DNA is copied without errors. When this is right, the number of microsatellite repeats is the same in all the cells. The MMR process relies on four main proteins: MLH1, MSH2, MSH6, and PMS2 that work together to repair mistakes in the DNA.

In some cancer patients, the MMR process works well. For these people, the cancer cells are proficient in mismatch repair (pMMR) and their tumor cells have the same number of repeats as in their healthy cells. This is also referred to as microsatellite stable or MSS.

In some embodiments, the colorectal cancer has a mutation, chromosomal change, or translocation which affects one or more of the WNT/beta-catenin, MAPK/PBK, TGF-β, or TP53 pathways. In some embodiments, the colorectal cancer has a mutation in a gene selected from c-MYC, KRAS, NRAS, HRAS, BRAF, PIK3CA, PTEN, SMAD2, or SMAD4. In some embodiments, the mCRC has a BRAF or KRAS mutation. In some embodiments, the BRAF mutation is V600E substitution mutation. In some embodiments, the KRAS mutation is a V9, Gl2, Gl3, Vl4, Ll9, Q22, D33, A59, G60, Q61 R68, Kll7, A146, R164, K176, or K180 substitution. In some embodiments, the KRAS mutation is a G12D, G12V, G12C, G12A, G12S or other G12 variants. In other embodiments, the KRAS mutation is G13D, G12R, Q61H, Q61P, Q61K, Q61R, Q61L, Q61H, R68S, A11_G12dup, Y71C, P34L, L19F, Q22K, A59T, A146V, K117N, A146T, G13C, Q61E, E98* and A155D. In some embodiments, the NRAS mutation is selected from Gl2, Gl3, G60, Q61, El23, or P185. In other embodiments, the NRAS mutation is selected from G12D, G13D, G13R, G12C, Q61L, Q61K, Q61H, Q61R, G12V, G12A, E132K, R164C and E76K. In some embodiments, the HRAS mutation is Gl2, Gl3, Q61, Kl17, R164, or P 167. In some embodiments, the colorectal cancer is KRAS wild-type. In some embodiments, the colorectal cancer is BRAF wild-type. In another embodiment, the colorectal cancer does not have a BRAFV600E mutation.

### Evaluating Levels of DKK1 in Tumor, Serum and Plasma

In a further embodiment, the subject's tumor (subject suffering from colorectal cancer) has detectable levels of DKK1 expression, as determined by one or more of the various standard mRNA or protein detection methods known in the art, e.g., chromogenic in situ hybridization (RNAscope), immunohistochemistry, qPCR, RNA-Seq and NanoString.

### H-Score and % Positive Values

The level of expression of a gene product of interest, e.g., the expression of DKK1, can be evaluated by methods of immunohistochemistry or chromogenic in situ hybridization techniques. Convenient semiquantitative measures of the level of expression are computing % positive value (% of tumor cells stained by DKK-1 RNA detecting reagent) or assigning an H-score (or “histo” score) to tumor samples. For H-score, a staining amount or intensity (0, 1+, 2+, or 3+) is determined for each cell in a fixed field. For in situ hybridization techniques the number of dots for each cell may be determined where 0 is no detected dots per cell, 1+ is 1-3 dots per cell, 2+ is 4-9 dots per cell and 3+ is 10+ dots per cell. The H-score may then be based on a predominant staining amount (or dot number per cell), or more complexly, can include the sum of individual percentages for each staining amount (or dot number per cell) level seen. By one method, the percentage of cells at each staining intensity (or dot number per cell) level is calculated, and finally, an H-score is assigned using the following formula:

H-score=[1×(% cells 1+)+2×(% cells 2+)+3×(% cells 3+)]

The final H-score, ranging from 0 to 300, gives more relative weight to higher-intensity or amount of staining (e.g., dots per cell) in a given tumor sample. The sample can then be considered positive or negative on the basis of a specific discriminatory threshold. See, for example, Hirsch F R, Varella-Garcia M, Bunn P A Jr, et al: Epidermal growth factor receptor in non-small-cell lung carcinomas: Correlation between gene copy number and protein expression and impact on prognosis. J Clin Oncol 21:3798-3807, 2003; and John T, Liu G, Tsao M-S: Overview of molecular testing in non-small-cell lung cancer: Mutational analysis, gene copy number, protein expression and other biomarkers of EGFR for the prediction of response to tyrosine kinase inhibitors. Oncogene 28:S14-S23, 2009.

In various embodiments, an H-score (e.g., a predetermined value of the H-score) can be from 0 to 300, for example, 0, 1, 2, 3 etc. Example predetermined values of H-score are: 1, 2, 3, 4, and 5. In certain embodiments, the predetermined value of H-score is: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 103, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300.

In alternative embodiments, the measure of DKK1 expression can be a value of the fraction of tumor cells that stain positive for DKK1 (% positive or tumor percent score (TPS)). First a staining amount (0, 1+, 2+, or 3+), based on number of dots in the cell, is determined for each tumor cell in a fixed field. After all neoplastic cells were assigned as “positive,” (e.g., detecting a single staining dot for RNAscope chromogentic in situ hybridization), the percentage of positive tumor cells is determined by adding up the total neoplastic cells with staining and dividing by the total number of neoplastic cells. % Positive can range from 0 to 100.

In various embodiments, % Positive value (e.g., a predetermined value of % positive) can be from 0% to 50%, for example from 1% to 5%. Example predetermined values of % Positive are: 1% or greater, 2% or greater, 3% or greater, 4% or greater, or 5% of greater. In certain embodiments, the predetermined value of % positive is: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

### RNAscope Analysis

One of the methods of computing an H-score of a sample is an RNAscope® in situ hybridization technique developed by and commercially available from Advanced Cell Diagnostics, Inc., as described, for example, at the URL https://acdbio.com/and from Flagship Biosciences, Broomfield, CO. This technique relies on an optical signal from a hybridization probe cognate to the mRNA of interest. The signal can be detected either by a bright-field or epifluorescent microscopy. The technique permits detection of a single molecule. See, for example, RNAscope: A Novel In Situ RNA Analysis Platform for Formalin-Fixed Paraffin-Embedded Tissues. Wang F, Flanagan J, Su N, Wang L C, Bui S, Nielson A, Wu X, Vo H T, Ma X J, Luo Y (2012). J of Mol Diagnostics, 14(1):22-29.

### Enzyme-Linked Immunosorbent Assay

Serum and plasma DKK1 levels can be measured using a modified immunoassay with electrochemiluminescent detection. In this assay, serum or plasma samples containing DKK1 is captured by anti-hDKK1 (MAB 1096) coated on microtiter plates. Unbound material is washed away leaving only captured DKK1. DKK1 is released from MAB 1096 coated plates by acidification (leaving MAB 1096 bound to the plate). The released DKK1 is transferred to a MSD plate where DKK1 is allowed to bind directly to the plate. The bound DKK1 is detected by addition of biotinylated anti-hDKK1 (MAB 1096) and subsequent development with streptavidin-ruthenium and MSD Read Buffer. Results are reported as concentration of DKK1 (ng/ml) in the serum or plasma samples. DKK1 AND DKK1 ANTIBODIES

Dickkopf-1 (DKK1) is a secreted modulator of Wnt signaling pathways, which influences a number of biological processes such as stem cell maintenance, cell fate decisions, cell proliferation, survival, migration and polarity determination during development and adult tissue homeostasis. DKK1 has been most extensively characterized as an inhibitor of canonical Wnt/beta-catenin dependent signaling and this has been associated with contributing to an immune suppressive tumor microenvironment. DKK1 has also been implicated in promoting tumor growth and metastasis through activation of noncanonical (beta-catenin independent Wnt signaling) and PI3K/AKT signaling pathways. DKK1 also regulates bone homeostasis during development and in adult organisms. DKK1 inhibits osteoblastogenesis, or the differentiation of mesechymal stem cells to osteoblasts (OB), a process promoted by Wnt signaling. As a result, the OB (bone formation)/osteoclast (OC) (bone resorption) equilibrium is shifted toward increased bone resorption, eventually resulting in osteolytic lesions. Certain tumors are associated with the development of osteolytic bone disease mediated by increased OC bone resorption and impaired OB bone formation.

In a further embodiment, the colorectal cancer has increased levels of DKK1 expression, as determined by one or more of the various standard mRNA or protein detection methods known in the art, e.g., chromogenic in situ hybridzidation or immunohistochemistry.

DKK1 antibodies have been described previously (see, e.g., U.S. Pat. No. 8,148,498, incorporated by reference herein in its entirety). The present DKK1 antibodies of the disclosure are therapeutically useful DKK1 antagonists possessing a number of desirable properties. For example, the DKK1 antibodies reduce DKK1 mediated inhibition of alkaline phosphatase, a marker of osteoblast activity, as well as treat various types of cancer (e.g., non-small cell lung cancer).

A full-length antibody as it exists naturally is an immunoglobulin molecule comprising 2 heavy (H) chains and 2 light (L) chains interconnected by disulfide bonds. The amino terminal portion of each chain includes a variable region of about 100-110 amino acids primarily responsible for antigen recognition via the complementarity determining regions (CDRs) contained therein. The carboxy-terminal portion of each chain defines a constant region primarily responsible for effector function.

The CDRs are interspersed with regions that are more conserved, termed framework regions (“FR”). Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the LCVR and HCVR regions is in accordance with the well-known Kabat numbering convention.

Light chains are classified as kappa or lambda, and are characterized by a particular constant region as known in the art. Heavy chains are classified as gamma, mu, alpha, delta, or epsilon, and define the isotype of an antibody as IgG, IgM, IgA, IgD, or IgE, respectively. IgG antibodies can be further divided into subclasses, e.g., IgG1, IgG2, IgG3, IgG4. Each heavy chain type is characterized by a particular constant region with a sequence well known in the art.

As used herein, the term “monoclonal antibody” (Mab) refers to an antibody that is derived from a single copy or clone including, for example, any eukaryotic, prokaryotic, or phage clone, and not the method by which it is produced. Mabs of the present invention preferably exist in a homogeneous or substantially homogeneous population. Complete Mabs contain 2 heavy chains and 2 light chains.

Unless specified otherwise, the term “DKK1 antibody” encompasses both a full-length antibody as well as an antigen binding-fragment of the DKK1 antibody.

“Antigen-binding fragments” of such monoclonal antibodies include, for example, Fab fragments, Fab′ fragments, F(ab′)₂ fragments, and single chain Fv fragments as well as bispecific and/or multivalent antibodies that may utilize the DKK1 antibody CDRs. Monoclonal antibodies and antigen-binding fragments thereof can be produced, for example, by recombinant technologies, phage display technologies, synthetic technologies, e.g., CDR-grafting, or combinations of such technologies, or other technologies known in the art. For example, mice can be immunized with human DKK1 or fragments thereof, the resulting antibodies can be recovered and purified, and determination of whether they possess binding and functional properties similar to or the same as the antibody compounds disclosed herein can be assessed by the methods known in the art. Antigen-binding fragments can also be prepared by conventional methods. Methods for producing and purifying antibodies and antigen-binding fragments are well known in the art and can be found, for example, in Harlow and Lane (1988) Antibodies. A Laboratory Manual, Cold Spring Harbor Laboratory Press, Cold Spring Harbor, N.Y., chapters 5-8 and 15, ISBN 0-87969-314-2.

Monoclonal DKK1 antibodies disclosed herein are engineered to comprise framework regions that are substantially human or fully human surrounding CDRs derived from a non-human antibody. “Antigen-binding fragments” of such human engineered antibodies include, for example, Fab fragments, Fab′ fragments, F(ab′)₂ fragments, and single chain Fv fragments. “Framework region” or “framework sequence” refers to any one of framework regions 1 to 4. Human engineered antibodies and antigen-binding fragments thereof encompassed by the antibodies disclosed herein include molecules wherein any one or more of framework regions 1 to 4 is substantially or fully human, i.e., wherein any of the possible combinations of individual substantially or fully human framework regions 1 to 4, is present. For example, this includes molecules in which framework region 1 and framework region 2, framework region 1 and framework region 3, framework region 1, 2, and 3, etc., are substantially or fully human. Substantially human frameworks are those that have at least about 80% sequence identity to a known human germline framework sequence. Preferably, the substantially human frameworks have at least about 85%, about 90%, about 95%, or about 99% sequence identity to a known human germline framework sequence.

Human engineered antibodies in addition to those disclosed herein exhibiting similar functional properties can be generated using several different methods. The specific antibody compounds disclosed herein can be used as templates or parent antibody compounds to prepare additional antibody compounds. In one approach, the parent antibody compound CDRs are grafted into a human framework that has a high sequence identity with the parent antibody compound framework. The sequence identity of the new framework will generally be at least about 80%, at least about 85%, at least about 90%, at least about 95%, or at least about 99% identical to the sequence of the corresponding framework in the parent antibody compound. This grafting may result in a reduction in binding affinity compared to that of the parent antibody. If this is the case, the framework can be back-mutated to the parent framework at certain positions based on specific criteria disclosed by Queen et al. (1991) Proc. Natl. Acad. Sci. USA 88:2869. Additional references describing methods useful in humanizing mouse antibodies include U.S. Pat. Nos. 4,816,397; 5,225,539, and 5,693,761; computer programs ABMOD and ENCAD as described in Levitt (1983) J. Mol. Biol. 168:595-620; and the method of Winter and co-workers (Jones et al. (1986) Nature 321:522-525; Riechmann et al. (1988) Nature 332:323-327; and Verhoeven et al. (1988) Science 239:1534-1536). Methods for identifying residues to consider for back-mutation are known in the art (see, e.g., U.S. Pat. No. 8,148,498).

The DKK1 antibody administered in the method of treatment described herein comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3.

In one embodiment, the DKK1 antibody comprises a LCDR1 having the amino sequence of SEQ ID NO:1, LCDR2 having the amino sequence of SEQ ID NO:2, LCDR3 having the amino sequence of SEQ ID NO:3, HCDR1 having the amino sequence of SEQ ID NO: 4, HCDR2 having the amino sequence of SEQ ID NO:5, and HCDR3 having the amino sequence of SEQ ID NO:6.

In another embodiment, the DKK1 antibody comprises a LCVR having the amino acid sequence of SEQ ID NO: 7 and a HCVR having the amino acid sequence of SEQ ID NO: 8. In a particular embodiment, the LCVR comprises the amino acid sequence of SEQ ID NO: 11 and the HCVR comprises the amino acid sequence of SEQ ID NO: 12.

In further embodiments, the DKK1 antibody comprises a heavy chain (HC) having the amino acid sequence of SEQ ID NO: 17 and a light chain (LC) having the amino acid sequence of SEQ ID NO: 18. The DKK1 antibody or antigen binding-fragment thereof comprising the HC and LC amino acid sequence of SEQ ID NO: 17 and SEQ ID NO: 18, respectively, is referred to herein as DKN-01. In particular, DKN-01 has the molecular/empirical formula C₆₃₉₄ H₉₈₁₀ N₁₆₉₈ O₂₀₁₂ S₄₂ and a molecular weight of 144015 Daltons (intact).

In certain embodiments, the DKK1 antibody disclosed herein is an IgG₄ antibody with a neutralizing activity against human DKK1 comprising the sequence set forth in SEQ ID NO: 22, or a fragment thereof. For example, canonical Wnt signaling is important for osteoblast differentiation and activity. Wnt-3a combined with BMP-4 induces multipotent mouse C2C12 cells to differentiate into osteoblasts with a measurable endpoint of alkaline phosphatase (“AP”), a marker of osteoblast activity. DKK1, an inhibitor of canonical Wnt signaling, inhibits the differentiation and production of AP. Neutralizing DKK1 antibodies prevent DKK1-mediated inhibition of AP. Antibodies which block DKK1 inhibitory activity prevent the loss of AP activity (see U.S. Pat. No. 8,148,498). In a particular embodiment, the DKK1 antibody possessing neutralizing activity is DKN-01, which is an IgG₄ antibody.

The DKK1 antibodies disclosed herein possess high affinity (Kd) to DKK1 (e.g., human DKK1, SEQ ID NO: 22), as described in U.S. Pat. No. 8,148,498. For example, the present DKK1 antibodies possess a Kd of between 0.5×10⁻¹² M and 3.0×10⁻¹¹ M, at 37° C.

### SEQUENCES

The following are sequences of the DKN-01 antibody that can be employed in the practice of the various example embodiments described herein.

LCDR1
(SEQ ID NO: 1)
His Ala Ser Asp Ser Ile Ser Asn Ser Leu His
LCDR2
(SEQ ID NO: 2)
Tyr Xaa Arg Gln Ser Xaa Gln
wherein Xaa at position 2 is Gly or Ala; and Xaa at position 6 is Ile or Glu
LCDR3
(SEQ ID NO: 3)
Gln Gln Ser Xaa Ser Trp Pro Leu His
wherein Xaa at position 4 is Glu or Ala
HCDR1
(SEQ ID NO: 4)
Gly Phe Thr Phe Ser Ser Tyr Thr Met Ser
HCDR2
(SEQ ID NO: 5)
Thr Ile Ser Gly Gly Gly Phe Gly Thr Tyr Tyr Pro Asp Ser Val Lys
HCDR3
(SEQ ID NO: 6)
Pro Gly Tyr Xaa Asn Tyr Tyr Phe Asp Ile
wherein Xaa at position 4 is His or Asn
LCVR
(SEQ ID NO: 7)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Xaa Arg Gln Ser Xaa Gln Gly Ile Pro Ala Arg Phe
Ser Gly Ser Gly Ser Gly Thr Asp Phe Thr Leu Thr Ile Ser Ser Leu Glu Pro Glu Asp Phe
Ala Val Tyr Tyr Cys Gln Gln Ser Xaa Ser Trp Pro Leu His Phe Gly Gly Gly Thr Lys Val
Glu Ile Lys
wherein Xaa at position 51 is Gly or Ala; Xaa at position 55 is Ile or Glu and Xaa at
position 92 is Glu or Ala.
HCVR
(SEQ ID NO: 8)
Glu Val Gln Leu Val Glu Ser Gly Gly Gly Leu Val Gln Pro Gly Gly Ser Leu
Arg Leu Ser Cys Ala Ala Ser Gly Phe Thr Phe Ser Ser Tyr Thr Met Ser Trp Val Arg Gln
Ala Pro Gly Lys Gly Leu Glu Trp Val Ala Thr Ile Ser Gly Gly Gly Phe Gly Thr Tyr Tyr
Pro Asp Ser Val Lys Gly Arg Phe Thr Ile Ser Arg Asp Asn Ala Lys Asn Ser Leu Tyr Leu
Gln Met Asn Ser Leu Arg Ala Glu Asp Thr Ala Val Tyr Tyr Cys Ala Arg Pro Gly Tyr Xaa
Asn Tyr Tyr Phe Asp Ile Trp Gly Gln Gly Thr Thr Val Thr Val Ser Ser
wherein Xaa at position 102 is His or Asn
LCVR
(SEQ ID NO: 9)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Glu Ser Trp Pro Leu His Phe Gly Gly Gly Thr Lys Val Glu
Ile Lys
HCVR
(SEQ ID NO: 10)
Glu Val Gln Leu Val Glu Ser Gly Gly Gly Leu Val Gln Pro Gly Gly Ser Leu
Arg Leu Ser Cys Ala Ala Ser Gly Phe Thr Phe Ser Ser Tyr Thr Met Ser Trp Val Arg Gln
Ala Pro Gly Lys Gly Leu Glu Trp Val Ala Thr Ile Ser Gly Gly Gly Phe Gly Thr Tyr Tyr
Pro Asp Ser Val Lys Gly Arg Phe Thr Ile Ser Arg Asp Asn Ala Lys Asn Ser Leu Tyr Leu
Gln Met Asn Ser Leu Arg Ala Glu Asp Thr Ala Val Tyr Tyr Cys Ala Arg Pro Gly Tyr His
Asn Tyr Tyr Phe Asp Ile Trp Gly Gln Gly Thr Thr Val Thr Val Ser Ser
LCVR
(SEQ ID NO: 11)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Glu Ser Trp Pro Leu His Phe Gly Gly Gly Thr Lys Val Glu
Ile Lys
HCVR
(SEQ ID NO: 12)
Glu Val Gln Leu Val Glu Ser Gly Gly Gly Leu Val Gln Pro Gly Gly Ser Leu
Arg Leu Ser Cys Ala Ala Ser Gly Phe Thr Phe Ser Ser Tyr Thr Met Ser Trp Val Arg Gln
Ala Pro Gly Lys Gly Leu Glu Trp Val Ala Thr Ile Ser Gly Gly Gly Phe Gly Thr Tyr Tyr
Pro Asp Ser Val Lys Gly Arg Phe Thr Ile Ser Arg Asp Asn Ala Lys Asn Ser Leu Tyr Leu
Gln Met Asn Ser Leu Arg Ala Glu Asp Thr Ala Val Tyr Tyr Cys Ala Arg Pro Gly Tyr Asn
Asn Tyr Tyr Phe Asp Ile Trp Gly Gln Gly Thr Thr Val Thr Val Ser Ser
LCVR
(SEQ ID NO: 13)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Ala Ser Trp Pro Leu His Phe Gly Gly Gly Thr Lys Val Glu
Ile Lys
LCVR
(SEQ ID NO: 14)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Glu Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Ala Ser Trp Pro Leu His Phe Gly Gly Gly Thr Lys Val Glu
Ile Lys
HC
(SEQ ID NO: 15)
Glu Val Gln Leu Val Glu Ser Gly Gly Gly Leu Val Gln Pro Gly Gly Ser Leu
Arg Leu Ser Cys Ala Ala Ser Gly Phe Thr Phe Ser Ser Tyr Thr Met Ser Trp Val Arg Gln
Ala Pro Gly Lys Gly Leu Glu Trp Val Ala Thr Ile Ser Gly Gly Gly Phe Gly Thr Tyr Tyr
Pro Asp Ser Val Lys Gly Arg Phe Thr Ile Ser Arg Asp Asn Ala Lys Asn Ser Leu Tyr Leu
Gln Met Asn Ser Leu Arg Ala Glu Asp Thr Ala Val Tyr Tyr Cys Ala Arg Pro Gly Tyr His
Asn Tyr Tyr Phe Asp Ile Trp Gly Gln Gly Thr Thr Val Thr Val Ser Ser Ala Ser Thr Lys
Gly Pro Ser Val Phe Pro Leu Ala Pro Cys Ser Arg Ser Thr Ser Glu Ser Thr Ala Ala Leu
Gly Cys Leu Val Lys Asp Tyr Phe Pro Glu Pro Val Thr Val Ser Trp Asn Ser Gly Ala Leu
Thr Ser Gly Val His Thr Phe Pro Ala Val Leu Gln Ser Ser Gly Leu Tyr Ser Leu Ser Ser Val
Val Thr Val Pro Ser Ser Ser Leu Gly Thr Lys Thr Tyr Thr Cys Asn Val Asp His Lys Pro
Ser Asn Thr Lys Val Asp Lys Arg Val Glu Ser Lys Tyr Gly Pro Pro Cys Pro Pro Cys Pro
Ala Pro Glu Ala Ala Gly Gly Pro Ser Val Phe Leu Phe Pro Pro Lys Pro Lys Asp Thr Leu
Met Ile Ser Arg Thr Pro Glu Val Thr Cys Val Val Val Asp Val Ser Gln Glu Asp Pro Glu
Val Gln Phe Asn Trp Tyr Val Asp Gly Val Glu Val His Asn Ala Lys Thr Lys Pro Arg Glu
Glu Gln Phe Asn Ser Thr Tyr Arg Val Val Ser Val Leu Thr Val Leu His Gln Asp Trp Leu
Asn Gly Lys Glu Tyr Lys Cys Lys Val Ser Asn Lys Gly Leu Pro Ser Ser Ile Glu Lys Thr
Ile Ser Lys Ala Lys Gly Gln Pro Arg Glu Pro Gln Val Tyr Thr Leu Pro Pro Ser Gln Glu
Glu Met Thr Lys Asn Gln Val Ser Leu Thr Cys Leu Val Lys Gly Phe Tyr Pro Ser Asp Ile
Ala Val Glu Trp Glu Ser Asn Gly Gln Pro Glu Asn Asn Tyr Lys Thr Thr Pro Pro Val Leu
Asp Ser Asp Gly Ser Phe Phe Leu Tyr Ser Arg Leu Thr Val Asp Lys Ser Arg Trp Gln Glu
Gly Asn Val Phe Ser Cys Ser Val Met His Glu Ala Leu His Asn His Tyr Thr Gln Lys Ser
Leu Ser Leu Ser Leu Gly
LC
(SEQ ID NO: 16)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Glu Ser Trp Pro Leu His Phe Gly Gly Gly Thr Lys Val Glu
Ile Lys Arg Thr Val Ala Ala Pro Ser Val Phe Ile Phe Pro Pro Ser Asp Glu Gln Leu Lys Ser
Gly Thr Ala Ser Val Val Cys Leu Leu Asn Asn Phe Tyr Pro Arg Glu Ala Lys Val Gln Trp
Lys Val Asp Asn Ala Leu Gln Ser Gly Asn Ser Gln Glu Ser Val Thr Glu Gln Asp Ser Lys
Asp Ser Thr Tyr Ser Leu Ser Ser Thr Leu Thr Leu Ser Lys Ala Asp Tyr Glu Lys His Lys
Val Tyr Ala Cys Glu Val Thr His Gln Gly Leu Ser Ser Pro Val Thr Lys Ser Phe Asn Arg
Gly Glu Cys
HC
(SEQ ID NO: 17)
Glu Val Gln Leu Val Glu Ser Gly Gly Gly Leu Val Gln Pro Gly Gly Ser Leu
Arg Leu Ser Cys Ala Ala Ser Gly Phe Thr Phe Ser Ser Tyr Thr Met Ser Trp Val Arg Gln
Ala Pro Gly Lys Gly Leu Glu Trp Val Ala Thr Ile Ser Gly Gly Gly Phe Gly Thr Tyr Tyr
Pro Asp Ser Val Lys Gly Arg Phe Thr Ile Ser Arg Asp Asn Ala Lys Asn Ser Leu Tyr Leu
Gln Met Asn Ser Leu Arg Ala Glu Asp Thr Ala Val Tyr Tyr Cys Ala Arg Pro Gly Tyr Asn
Asn Tyr Tyr Phe Asp Ile Trp Gly Gln Gly Thr Thr Val Thr Val Ser Ser Ala Ser Thr Lys
Gly Pro Ser Val Phe Pro Leu Ala Pro Cys Ser Arg Ser Thr Ser Glu Ser Thr Ala Ala Leu
Gly Cys Leu Val Lys Asp Tyr Phe Pro Glu Pro Val Thr Val Ser Trp Asn Ser Gly Ala Leu
Thr Ser Gly Val His Thr Phe Pro Ala Val Leu Gln Ser Ser Gly Leu Tyr Ser Leu Ser Ser Val
Val Thr Val Pro Ser Ser Ser Leu Gly Thr Lys Thr Tyr Thr Cys Asn Val Asp His Lys Pro
Ser Asn Thr Lys Val Asp Lys Arg Val Glu Ser Lys Tyr Gly Pro Pro Cys Pro Pro Cys Pro
Ala Pro Glu Ala Ala Gly Gly Pro Ser Val Phe Leu Phe Pro Pro Lys Pro Lys Asp Thr Leu
Met Ile Ser Arg Thr Pro Glu Val Thr Cys Val Val Val Asp Val Ser Gln Glu Asp Pro Glu
Val Gln Phe Asn Trp Tyr Val Asp Gly Val Glu Val His Asn Ala Lys Thr Lys Pro Arg Glu
Glu Gln Phe Asn Ser Thr Tyr Arg Val Val Ser Val Leu Thr Val Leu His Gln Asp Trp Leu
Asn Gly Lys Glu Tyr Lys Cys Lys Val Ser Asn Lys Gly Leu Pro Ser Ser Ile Glu Lys Thr
Ile Ser Lys Ala Lys Gly Gln Pro Arg Glu Pro Gln Val Tyr Thr Leu Pro Pro Ser Gln Glu
Glu Met Thr Lys Asn Gln Val Ser Leu Thr Cys Leu Val Lys Gly Phe Tyr Pro Ser Asp Ile
Ala Val Glu Trp Glu Ser Asn Gly Gln Pro Glu Asn Asn Tyr Lys Thr Thr Pro Pro Val Leu
Asp Ser Asp Gly Ser Phe Phe Leu Tyr Ser Arg Leu Thr Val Asp Lys Ser Arg Trp Gln Glu
Gly Asn Val Phe Ser Cys Ser Val Met His Glu Ala Leu His Asn His Tyr Thr Gln Lys Ser
Leu Ser Leu Ser Leu Gly
LC
(SEQ ID NO: 18)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Glu Ser Trp Pro Leu His Phe Gly Gly Gly Thr Lys Val Glu
Ile Lys Arg Thr Val Ala Ala Pro Ser Val Phe Ile Phe Pro Pro Ser Asp Glu Gln Leu Lys Ser
Gly Thr Ala Ser Val Val Cys Leu Leu Asn Asn Phe Tyr Pro Arg Glu Ala Lys Val Gln Trp
Lys Val Asp Asn Ala Leu Gln Ser Gly Asn Ser Gln Glu Ser Val Thr Glu Gln Asp Ser Lys
Asp Ser Thr Tyr Ser Leu Ser Ser Thr Leu Thr Leu Ser Lys Ala Asp Tyr Glu Lys His Lys
Val Tyr Ala Cys Glu Val Thr His Gln Gly Leu Ser Ser Pro Val Thr Lys Ser Phe Asn Arg
Gly Glu Cys
HC
(SEQ ID NO: 19)
Glu Val Gln Leu Val Glu Ser Gly Gly Gly Leu Val Gln Pro Gly Gly Ser Leu
Arg Leu Ser Cys Ala Ala Ser Gly Phe Thr Phe Ser Ser Tyr Thr Met Ser Trp Val Arg Gln
Ala Pro Gly Lys Gly Leu Glu Trp Val Ala Thr Ile Ser Gly Gly Gly Phe Gly Thr Tyr Tyr
Pro Asp Ser Val Lys Gly Arg Phe Thr Ile Ser Arg Asp Asn Ala Lys Asn Ser Leu Tyr Leu
Gln Met Asn Ser Leu Arg Ala Glu Asp Thr Ala Val Tyr Tyr Cys Ala Arg Pro Gly Tyr His
Asn Tyr Tyr Phe Asp Ile Trp Gly Gln Gly Thr Thr Val Thr Val Ser Ser Ala Ser Thr Lys
Gly Pro Ser Val Phe Pro Leu Ala Pro Cys Ser Arg Ser Thr Ser Glu Ser Thr Ala Ala Leu
Gly Cys Leu Val Lys Asp Tyr Phe Pro Glu Pro Val Thr Val Ser Trp Asn Ser Gly Ala Leu
Thr Ser Gly Val His Thr Phe Pro Ala Val Leu Gln Ser Ser Gly Leu Tyr Ser Leu Ser Ser Val
Val Thr Val Pro Ser Ser Ser Leu Gly Thr Lys Thr Tyr Thr Cys Asn Val Asp His Lys Pro
Ser Asn Thr Lys Val Asp Lys Arg Val Glu Ser Lys Tyr Gly Pro Pro Cys Pro Pro Cys Pro
Ala Pro Glu Ala Ala Gly Gly Pro Ser Val Phe Leu Phe Pro Pro Lys Pro Lys Asp Thr Leu
Met Ile Ser Arg Thr Pro Glu Val Thr Cys Val Val Val Asp Val Ser Gln Glu Asp Pro Glu
Val Gln Phe Asn Trp Tyr Val Asp Gly Val Glu Val His Asn Ala Lys Thr Lys Pro Arg Glu
Glu Gln Phe Asn Ser Thr Tyr Arg Val Val Ser Val Leu Thr Val Leu His Gln Asp Trp Leu
Asn Gly Lys Glu Tyr Lys Cys Lys Val Ser Asn Lys Gly Leu Pro Ser Ser Ile Glu Lys Thr
Ile Ser Lys Ala Lys Gly Gln Pro Arg Glu Pro Gln Val Tyr Thr Leu Pro Pro Ser Gln Glu
Glu Met Thr Lys Asn Gln Val Ser Leu Thr Cys Leu Val Lys Gly Phe Tyr Pro Ser Asp Ile
Ala Val Glu Trp Glu Ser Asn Gly Gln Pro Glu Asn Asn Tyr Lys Thr Thr Pro Pro Val Leu
Asp Ser Asp Gly Ser Phe Phe Leu Tyr Ser Arg Leu Thr Val Asp Lys Ser Arg Trp Gln Glu
Gly Asn Val Phe Ser Cys Ser Val Met His Glu Ala Leu His Asn His Tyr Thr Gln Lys Ser
Leu Ser Leu Ser Leu Gly
LC
(SEQ ID NO: 20)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Gly Arg Gln Ser Ile Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Ala Ser Trp Pro Leu His Phe Gly Gly Gly Thr Lys Val Glu
Ile Lys Arg Thr Val Ala Ala Pro Ser Val Phe Ile Phe Pro Pro Ser Asp Glu Gln Leu Lys Ser
Gly Thr Ala Ser Val Val Cys Leu Leu Asn Asn Phe Tyr Pro Arg Glu Ala Lys Val Gln Trp
Lys Val Asp Asn Ala Leu Gln Ser Gly Asn Ser Gln Glu Ser Val Thr Glu Gln Asp Ser Lys
Asp Ser Thr Tyr Ser Leu Ser Ser Thr Leu Thr Leu Ser Lys Ala Asp Tyr Glu Lys His Lys
Val Tyr Ala Cys Glu Val Thr His Gln Gly Leu Ser Ser Pro Val Thr Lys Ser Phe Asn Arg
Gly Glu Cys
LC
(SEQ ID NO: 21)
Glu Ile Val Leu Thr Gln Ser Pro Ala Thr Leu Ser Leu Ser Pro Gly Glu Arg Ala
Thr Leu Ser Cys His Ala Ser Asp Ser Ile Ser Asn Ser Leu His Trp Tyr Gln Gln Lys Pro Gly
Gln Ala Pro Arg Leu Leu Ile Tyr Tyr Ala Arg Gln Ser Glu Gln Gly Ile Pro Ala Arg Phe Ser
Gly Ser Gly Ser Gly Thr Asp Phe Thr Leu Thr Ile Ser Ser Leu Glu Pro Glu Asp Phe Ala
Val Tyr Tyr Cys Gln Gln Ser Ala Ser Trp Pro Leu His Phe Gly Gly Gly Thr Lys Val Glu
Ile Lys Arg Thr Val Ala Ala Pro Ser Val Phe Ile Phe Pro Pro Ser Asp Glu Gln Leu Lys Ser
Gly Thr Ala Ser Val Val Cys Leu Leu Asn Asn Phe Tyr Pro Arg Glu Ala Lys Val Gln Trp
Lys Val Asp Asn Ala Leu Gln Ser Gly Asn Ser Gln Glu Ser Val Thr Glu Gln Asp Ser Lys
Asp Ser Thr Tyr Ser Leu Ser Ser Thr Leu Thr Leu Ser Lys Ala Asp Tyr Glu Lys His Lys
Val Tyr Ala Cys Glu Val Thr His Gln Gly Leu Ser Ser Pro Val Thr Lys Ser Phe Asn Arg
Gly Glu Cys
Human DKK1 Amino Acid Sequence
(SEQ ID NO: 22)
Thr Leu Asn Ser Val Leu Asn Ser Asn Ala Ile Lys Asn Leu Pro Pro Pro Leu Gly
Gly Ala Ala Gly His Pro Gly Ser Ala Val Ser Ala Ala Pro Gly Ile Leu Tyr Pro Gly Gly Asn
Lys Tyr Gln Thr Ile Asp Asn Tyr Gln Pro Tyr Pro Cys Ala Glu Asp Glu Glu Cys Gly Thr
Asp Glu Tyr Cys Ala Ser Pro Thr Arg Gly Gly Asp Ala Gly Val Gln Ile Cys Leu Ala Cys
Arg Lys Arg Arg Lys Arg Cys Met Arg His Ala Met Cys Cys Pro Gly Asn Tyr Cys Lys
Asn Gly Ile Cys Val Ser Ser Asp Gln Asn His Phe Arg Gly Glu Ile Glu Glu Thr Ile Thr Glu
Ser Phe Gly Asn Asp His Ser Thr Leu Asp Gly Tyr Ser Arg Arg Thr Thr Leu Ser Ser Lys
Met Tyr His Thr Lys Gly Gln Glu Gly Ser Val Cys Leu Arg Ser Ser Asp Cys Ala Ser Gly
Leu Cys Cys Ala Arg His Phe Trp Ser Lys Ile Cys Lys Pro Val Leu Lys Glu Gly Gln Val
Cys Thr Lys His Arg Arg Lys Gly Ser His Gly Leu Glu Ile Phe Gln Arg Cys Tyr Cys Gly
Glu Gly Leu Ser Cys Arg Ile Gln Lys Asp His His Gln Ala Ser Asn Ser Ser Arg Leu His
Thr Cys Gln Arg His
VEGF/VEGFR Inhibitors

Vascular endothelial growth factor (VEGF)/vascular endothelial growth factor receptor (VEGFR) inhibitors are agents that inhibit the activity of VEGF and VEGFR (including VEGFR2). Anti-VEGF monoclonal antibodies include bevacizumab (Avastin, Genentech) or bevacizumab-awwb (Mvasi, Amgen). Bevacizumab is vascular endothelial growth factor directed antibody. Bevacizumab is a recombinant humanized monoclonal IgGl antibody that contains human framework regions and murine complementarity-determining regions (CDRs). Bevacizumab binds VEGF and prevents the interaction of VEGF to its receptors (Flt-I and KDR) on the surface of endothelial cells. The interaction of VEGF with its receptors leads to endothelial cell proliferation and new blood vessel formation in in vitro models of angiogenesis. Administration of bevacizumab causes reduction of microvascular growth and inhibition of metastatic disease progression.

In various embodiments of the methods of the present invention, Bevacizumab can be replaced with one of its biosimilars. For example, bevacizumab can be replaced with Mvasi (bevacizumab-awwb), available from Amgen Inc. In another example, bevacizumab can be replaced with Zirabev (bevacizumab-bvzr), available from Pfizer Inc. In another example, bevacizumab can be replaced with Alymsys (bevacizumab-maly), available from Amneal Pharmaceuticals LLC. In another example, bevacizumab can be replaced with Vegzelma (bevacizumab-adcd), available from Celltrion Healthcare.

The mechanism of action of the biosimilars of bevacizumab is considered to be similar to bevacizumab.

In treating colorectal cancer, bevacizumab is generally administered on day 1 of each 14-day cycle of the induction phase and maintenance phase, or once every 2 weeks, as an intravenous injection at a dose of between about 4 and 12 mg/kg, with dosing adjustments as required. In some embodiments, bevacizumab is administered at about 5 mg/kg. In some embodiments, bevacizumab is administered at about 10 mg/kg.

Ramucirumab (Cyramza; Eli Lily) is a human vascular endothelial growth factor receptor 2 (VEGFR2) antagonist that specifically binds VEGFR2 and blocks binding of VEGFR ligands, VEGF-A, VEGF-C, and VEGF-D. As a result, ramucirumab inhibits ligand-stimulated activation of VEGFR2, thereby inhibiting ligand-induced proliferation, and migration of human endothelial cells. Ramucirumab is a recombinant human IgGl monoclonal antibody. Ramucirumab has an approximate molecular weight of 147 kDa. Ramucirumab is produced in genetically engineered mammalian NSO cells. Ramucirumab injection for intravenous use is a sterile, preservative-free, clear to slightly opalescent and colorless to slightly yellow solution. Ramucirumab is supplied at a concentration of 10 mg/mL in either 100 mg (10 mL) or 500 mg (50 mL) single-dose vials. Ramucirumab is formulated in glycine (9.98 mg/mL), histidine (0.65 mg/mL), histidine monohydrochloride (1.22 mg/mL), polysorbate 80 (0.1 mg/mL), sodium chloride (4.383 mg/mL), and Water for Injection, USP, pH 6.0.

In treating colorectal cancer, ramucirumab is generally administered on day 1 of each 14-day cycle of the induction and maintenance phase, or once every two weeks, as an intravenous infusion at a dose of between about 5 and 15 mg/kg, with dosing adjustments as required. In some embodiments, ramucirumab is administered at about 8 mg/kg as an intravenous infusion over 1 hour.

In various embodiments, the following VEGF and/or VEGFR inhibitors can be employed when practicing the methods disclosioed herein: Votrient (pazopanib, Novartis), Sutent (sunitinib, Pfizer), Nexavar (sorafenib, Bayer), Stivarga (regorafenib, Bayer), Cabometyx (cabozantinib, Exelixis), Lenvima (lenvatinib, Eisai), Iclusig (ponatinib, Ariad), Cometriq (cabozantinib, Exelixis), Zaltrap (ziv-aflibercept, Regeneron), Inlyta (axitinib, Pfizer), Fotivda (tivozanib, Aveo), Cyramza (ramucirumab, Eli Lilly), Caprelsa (vandetanib, Genzyme), or Alymsys (bevacizumab, Amneal).

### Chemotherapetic Agents

Many different kinds of chemotherapy or chemo drugs are used to treat cancer-either alone or in combination with other drugs or treatments. These drugs are very different in their chemical composition (what they are made of), how they are prescribed and given, how useful they are in treating certain types of cancer, and the side effects they might have.

Not all medicines and drugs to treat cancer work the same way. Other drugs to treat cancer, such as targeted therapy, hormone therapy, and immunotherapy work differently than traditional or standard chemotherapeutic agents. The chemotherapeutic agents used in the method of treatment described herein are traditional or standard chemotherapeutics agents and are not targeted therapy, hormone therapy or immunotherapy (e.g., biologics).

Chemotherapy drugs can target cells at different phases of the cell cycle. Chemotherapy drugs can be grouped by how they work, their chemical structure, and their relationships to other drugs. Some drugs work in more than one way, and may belong to more than one group.

Alkylating agents keep the cell from proliferating by damaging its DNA. Examples of alkylating agents for use as the at least one chemotherapeutic agent of the method described herein include: Altretamine; Bendamustine; Busulfan; Carboplatin; Carmustine; Chlorambucil; Cisplatin; Cyclophosphamide; Dacarbazine; Ifosfamide; Lomustine; Mechlorethamine; Melphalan; Oxaliplatin; Temozolomide; Thiotepa; and Trabectedin.

Antimetabolites interfere with DNA and RNA by acting as a substitute for the normal building blocks of RNA and DNA. When this happens, the DNA cannot make copies of itself, and a cell cannot divide. Examples of antimetabolites for use as the at least one chemotherapeutic agent of the method described herein include: Azacitidine; 5-fluorouracil (5-FU); 6-mercaptopurine (6-MP); Capecitabine; Cladribine; Clofarabine; Cytarabine (Ara-C); Decitabine; Floxuridine; Fludarabine; Gemcitabine; Hydroxyurea; Methotrexate; Nelarabine; Pemetrexed; Pentostatin; Pralatrexate; Thioguanine; and Trifluridine/tipiracil combination.

Anthracyclines are anti-tumor antibiotics that interfere with enzymes involved in copying DNA during the cell cycle. They bind with DNA so it cannot make copies of itself, and a cell cannot divide. Examples of anthracyclines for use as the at least one chemotherapeutic agent of the method described herein include: Daunorubicin; Doxorubicin; Doxorubicin liposomal; Epirubicin; Idarubicin; and Valrubicin. Anti-tumor antibiotics that are not anthracyclines include: Bleomycin; Dactinomycin; Mitomycin-C; and Mitoxantrone.

Topoisomerase inhibitors are drugs that interfere with enzymes called topoisomerases. Topoisomerase I inhibitors (also called camptothecins) include: Irinotecan; Irinotecan liposomal; and Topotecan. Topoisomerase II inhibitors (also called epipodophyllotoxins) include: Etoposide (VP-16); Mitoxantrone (also acts as an anti-tumor antibiotic); and Teniposide.

Mitotic inhibitors include taxanes and vinca alkaloids. Taxanes include: Cabazitaxel; Docetaxel; Nab-paclitaxel; and Paclitaxel. Vinca alkaloids include: Vinblastine; Vincristine; Vincristine liposomal; and Vinorelbine.

Some chemotherapy drugs do not fit well into any of the listed categories. Examples include: All-trans-retinoic acid; Arsenic trioxide; Asparaginase; Eribulin; Hydroxyurea; Ixabepilone; Mitotane; Omacetaxine; Pegaspargase; Procarbazine; Romidepsin; and Vorinostat.

In a particular embodiment, the one or more optional chemotherapeutic agents is a fluorouracil-based chemotherapy and is administered in accordance with the method described herein. Fluorouracil-based chemotherapies, include, but are not limited to: 5-fluorouracil (5-FU); combinations including 5-FU such as oxaliplatin plus 5-FU/leucovorin (FOLFOX), FLOT (5-FU/leucovorin, oxaliplatin, and docetaxel), Irinotecan plus 5-FU/leucovorin (FOLFIRI)

FOLFOX is a three-drug regimen composed of fluorouracil (5-FU), folinic acid (e.g., leucovorin or levoleucovorin), and oxaliplatin often used in the treatment of mCRC (National Comprehensive Cancer Network. NCCN Clinical Practice Guidelines in Oncology Colon Cancer. Version 2. 2019. NCCN, Fort Washington, PA). Versions of FOLFOX include FOLFOX4, FOLFOX6, modified FOLFOX6 (mFOLFOX6), FOLFOX7, and modified FOLFOX7. Descriptions of the FOLFOX versions are readily available in the literature and well-known.

In one embodiment, the FOLFOX dosing regimen consists of leucovorin or leucovorin, oxaliplatin and 5-fluorouracil and will be commercially sourced as part of standard of clinical care the administration regimen for modified FOLFOX6 (mFOLFOX6) can include folinic acid 400 mg/m2 IV on Day 1, fluorouracil 1200 mg/m2 IV/day (Days 1 and 2), and oxaliplatin 85 mg/m2 on D1 of each 14-day cycle. Additional information can be found in the current prescribing information.

In one embodiment, the FOLFIRI dosing regimen consists of leucovorin or leucovorin, irinotecan and 5-fluorouracil and will be commercially sourced as part of standard of clinical care. The administration regimen for FOLFIRI regimen can include a 90-min IV infusion of irinotecan (180 mg/m2) followed by a simplified LV5FU2 regimen (leucovorin [400 mg/m2] and bolus fluorouracil [400 mg/m2] on day 1 and a 46-h infusion of fluorouracil [2400 mg/m2]). Additional information can be found in the current prescribing information.

In a specific embodiment, the one of more chemotherapeutic therapeutics agents for use in the method of treating colorectal cancer is, mFOLFOX6 or FOLFIRI. TARGETED THERAPY (as additional therapeutic agents for co-administration) (PI3K Inhibitors)

Phosphoinositide 3-kinase inhibitors (PI3K inhibitors) are a class of drugs that function by inhibiting one or more of the phosphoinositide 3-kinase (PI3K) enzymes, which are part of the PI3K/AKT/mTOR pathway. This signal pathway regulates cellular functions such as growth and survival. It is strictly regulated in healthy cells, but is always active in many cancer cells, allowing the cancer cells to better survive and multiply. PI3K inhibitors block the PI3K/AKT/mTOR pathway and thus slow down cancer growth. They are examples of a targeted therapy.

PI3K inhibitors include, but are not limited to, copanlisib (Aliqopa), duvelisib (Copiktra), idelalisib (Zydelig) and alpelisib (Piqray).

In some alternative embodiments, the method described herein further includes the administration of a PI3K inhibitor and/or immune modulating agent. Examples of PI3K inhibitors include, but are not limited to, copanlisib (Aliqopa), duvelisib (Copiktra), idelalisib (Zydelig) and alpelisib (Pigras).

IMMUNOTHERAPY (as additional therapeutic agents for co-administration)

Combination with an Immune-Checkpoint Inhibitor

In some alternative embodiments, the method described herein further includes the administration of an immune-checkpoint inhibitor or immune modulating agent.

Examples of immune checkpoint inhibitors and immune modulating agents include, but are not limited to, a PD-1 inhibitor, PD-L1 inhibitor, PD-L2 inhibitor, CTLA-4 inhibitor. In certain aspects, the immune modulator is an antibody, such as a monoclonal antibody.

In some alternative embodiments, the immune checkpoint inhibitor is a PD-1 inhibitor that blocks the interaction of PD-1 and PD-L1 by binding to the PD-1 receptor, and in turn inhibits immune suppression. In some alternative embodiments, the immune checkpoint inhibitor is a PD-1 immune checkpoint inhibitor selected from, but not limited to, nivolumab (Opdivo®; Bristol-Myers Squibb), pembrolizumab (Keytruda®; Merck), pidilizumab, formerly CT-011, (Pfizer), AMP-224 (Amplimmune); sasanlimab (PF-06801591; Pfizer), spartalizumab (PDR001; Novartis), cemiplimab (Libtayo R; REGN2810; Regeneron), retifanlimab (MGA012 MacroGenics and Zynyz; Incyte), tislelizumab (BeiGene), camrelizumab (SHR-1210; Jiangsu Hengrui Medicine Company), dostarlimab (TSR-042; GlaxoSmithKline), budigalimab (ABBV-181; Abbvie) and zimberelimab (AB122; Arcus).

In some alternative embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab (Tecentriq®; Genentech), durvalumab (Imfinzi®; AstraZeneca); avelumab (Bavencio®; Merck), envafolimab (KN035; Alphamab), BMS-936559 (Bristol-Myers Squibb), lodapolimab (LY3300054; Eli Lilly), cosibelimab (Checkpoint Therapeutics), sugemalimab (Cstone Pharmaceuticals), and adebrelimab.

In one aspect, the immune checkpoint inhibitor is a CTLA-4 immune checkpoint inhibitor that binds to CTLA-4 and inhibits immune suppression. CTLA-4 inhibitors include, but are not limited to, ipilimumab (Yervoy R, Bristol Myers Squibb); tremelimumab (AstraZeneca/MedImmune), zalifrelimab (AGEN I 884; Agenus) and AGEN204 I (Agenus).

In certain aspects of the example embodiments, the PD-1/PD-L1-axis checkpoint inhibitor is selected from pembrolizumab, nivolumab, tislelizumab, budigalimab, zimerelimab, cemiplimab, atezolizumab, avelumab, and durvalumab. For example, the PD-1/PD-L1-axis checkpoint inhibitor is pembrolizumab, nivolumab, tislelizumab, budigalimab or atezolizumab.

### Administration and Dosing

### Modes of Administration

The DKK1 antibody and the VEGF or VEGFR inhibitor (e.g., bevacizumab) and the one or more optional chemotherapeutic agents used in the combination described herein (i.e., the components of the combination therapy) can be formulated separately or in combination for parenteral (e.g., intravenous), oral, transdermal, sublingual, buccal, rectal, intranasal, intrabronchial or intrapulmonary administration. In a particular embodiment, the DKK1 antibody (e.g., DKN-01) is administered intravenously. In another particular embodiment, the VEGF or VEGFR inhibitor (e.g., bevacizumab) is administered intravenously. In a further embodiment, the DKK1 antibody and the VEGF or VEGFR inhibitor are formulated in combination for intravenous administration.

For parenteral administration, one or more of the components of the combination therapy (e.g., DKK1 antibody, the VEGF or VEGFR inhibitor (e.g., bevacizumab) and the one or more optional chemotherapeutic agents) for use in the methods or compositions of the invention can be formulated for injection or infusion, for example, intravenous, intramuscular or subcutaneous injection or infusion, or for administration in a bolus dose and/or infusion (e.g., continuous infusion). Suspensions, solutions or emulsions in an oily or aqueous vehicle, optionally containing other formulatory agents such as suspending, stabilizing and/or dispersing agents can be used.

For oral administration one or more of the components of the combination therapy (e.g., DKK1 antibody, the VEGF or VEGFR inhibitor (e.g., bevacizumab) and the one or more optional chemotherapeutic agents) can be in the form of tablets or capsules prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated using suitable methods. Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, one or more of the components of the combination therapy (e.g., DKK1 antibody, the VEGF or VEGFR inhibitor (e.g., bevacizumab) and the one or more optional chemotherapeutic agents) for use in the methods or compositions of the invention can be in the form of tablets or lozenges formulated in a conventional manner.

For rectal administration, one or more of the components of the combination therapy (e.g., DKK1 antibody, the VEGF or VEGFR inhibitor (e.g., bevacizumab) and the one or more optional chemotherapeutic agents) for use in the methods or compositions of the invention can be in the form of suppositories.

For sublingual administration, tablets can be formulated in conventional manner.

For intranasal, intrabronchial or intrapulmonary administration, conventional formulations can be employed.

Further, one or more of the components of the combination therapy (e.g., DKK1 antibody, the VEGF or VEGFR inhibitor (e.g., bevacizumab) and the one or more optional chemotherapeutic agents) for use in the methods or compositions of the invention can be formulated in a sustained release preparation. For example, the one or more of the components can be formulated with a suitable polymer or hydrophobic material which provides sustained and/or controlled release properties to the active agent compound. As such, one or more components of the combination therapy for use in the method of the invention can be administered in the form of microparticles, for example, by injection or in the form of wafers or discs by implantation. Various methods of formulating controlled release drug preparations are known in the art.

Administration of one or more components of the combination therapy, or a pharmaceutically acceptable salt thereof, or a composition comprising one or more components of the combination therapy (or pharmaceutical salt thereof) of the invention useful to practice the methods described herein, can be continuous, hourly, four times daily, three time daily, twice daily, once daily, once every other day, twice weekly, once weekly, once every two weeks, once a month, or once every two months, or longer, or some other intermittent dosing regimen.

### Combination Therapy

As used herein, “co-administration”, co-administering” “in combination with” or “in conjunction with” refers to administration of one treatment modality in addition to at least one other treatment modality. As such, “in combination with” or “in conjunction with” refers to administration of one treatment modality before, during, or after administration of at least one other treatment modality to the individual.

The DKK1 antibody disclosed herein can be used for treating colorectal cancer in combination with a VEGF or VEGFR inhibitor (e.g., bevacizumab) and one or more optional chemotherapeutic agents. Such combination administration can be by means of a single dosage form which includes a DKK1 antibody, the VEGF or VEGFR inhibitor (e.g., bevacizumab) and the one or more optional chemotherapeutic agents, such single dosage form including a tablet, capsule, spray, inhalation powder, injectable liquid or the like. Alternatively, combination administration (e.g., co-administration) can be by means of administration of different dosage forms, with one dosage form containing a DKK1 antibody, another dosage form including the VEGF or VEGFR inhibitor (e.g., bevacizumab) and yet another dosage form including the one more optional chemotherapeutic agents (if more than one chemotherapeutic agent is used it is understood that these agents can be administered using the same or different dosage forms). For example, the DKK1 antibody (e.g., DKN-01) and the VEGF or VEGFR inhibitor (e.g., bevacizumab) can be administered together in a single dosage form (e.g., a single dosage form for intravenous administration) and the one or more optional chemotherapeutic agents can be administered in a different single dose by any other suitable means.

The components of the combination therapy can be administered in any order. For example, the VEGF or VEGFR inhibitor (e.g., bevacizumab) can be administered before, simultaneously with, or after the administration of a DKK1 antibody and before, simultaneously with, or after the administration of the one or more optional chemotherapeutic agents. Accordingly, the components of the combination therapy can be administered together in a single formulation or can be administered in separate formulations, e.g., either simultaneously or sequentially, or both. For example, if a DKK1 antibody and the VEGF or VEGFR inhibitor (e.g., bevacizumab) are administered sequentially in separate compositions, the DKK1 antibody can be administered before or after the VEGF or VEGFR inhibitor (e.g., bevacizumab). The duration of time between the administration of the components of the combination therapy will be easily determined by the administering physician.

Further, the components of the combination therapy may or may not be administered on similar dosing schedules. For example, the DKK1 antibody and the VEGF or VEGFR inhibitor (e.g., bevacizumab) can have different half-lives and/or act on different time-scales such that the DKK1 antibody is administered with greater frequency than the VEGF or VEGFR inhibitor (e.g., bevacizumab) or vice-versa. For example, the DKK1 antibody and the VEGF or VEGFR inhibitor (e.g., bevacizumab) can be administered together (e.g., in a single dosage or sequentially) on one day, followed by administration of only the one or more optional chemotherapeutic agents a set number of days later. The number of days in between administration of components of the combination therapy can be appropriately determined according to the safety and pharmacodynamics of each drug.

In a particular embodiment, the treatment period for the combination treatment is a 21-Day cycle which can be repeated until the patient is determined to not be gaining any clinical benefit from the combination therapy. In another particular embodiment, the treatment period for the combination treatment is a 14-Day cycle, which can be repeated until the patient is determined to no longer need treatment or to not be gaining any clinical benefit from the combination therapy. For example, the patient can undergo from about one cycle to about 30 cycles (e.g., 14-Day cycles) of treatment (e.g., 1, 2, 3, 4, 5, 6, 7, 8, 9, 10, 11, 12, 13, 14, 15, 16, 7, 18, 19, 20, 21, 22, 23, 24, 25, 26, 27, 28, 29, 30).

As used herein, an “effective amount” refers to an amount of the combination of therapeutic agents that is therapeutically or prophylactically sufficient to treat the target disorder. An effective amount will depend on the age, gender, and weight of the patient, the current medical condition of the patient, and the nature of the colorectal cancer being treated. Those of skill in the art will be able to determine appropriate dosages depending on these and other factors.

Suitable doses per administration for a DKK1 antibody include doses of about or greater than about 15 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, or about 3,000 mg. Each suitable dose can be administered over a period time deemed appropriate by a skilled practitioner. For example, each suitable dose can be administered over a period of about 30 minutes and up to about 1 hour, about 2 hours, about 3, hours, about 4 hours, about 5 hours, about 6 hours, about 7 hours, or about 8 hours. In a specific embodiment, a suitable doses for the DKK1 antibody (e.g., DKN-01) can from about 20 mg to about 1200 mg, such as from about 30 mg to about 600 mg, such as from about 50 mg to about 500 mg, such as from about 50 mg to about 300 mg (such as 50 mg, 75 mg, 100 mg, 125 mg, 150 mg, 175 mg, 200 mg, 225 mg, 250 mg, 275 mg, 300 mg, 400 mg, 500 mg, 600 mg, 700 mg, 800 mg, 900 mg, 1000 mg, 1100 mg or 1200 mg). The selected dose can be administered intravenously over a period of about 30 minutes to about 2 hours. In a particular embodiment, a suitable dose for DKK1 antibody can be about 300 mg administered over a period of about 30 minutes and up to about 2 hours. Another suitable dose for the DKK1 antibody can be about 600 mg administered over a period of about 30 minutes and up to about 2 hours. Another suitable dose for the DKK1 antibody (e.g., DKN-0) 1) can be about 400 mg administered IV over a period of about 30 minutes to about 2 hours. Administration of these doses over the recited period of time can be accomplished using an intravenous route. For example, the DKK1 antibody (e.g., DKN-01) can be dosed on Day 1 of a 21-day cycle and then repeated on Day 1 for every additional cycle. For such a 21-day cycle dosing can be in the amounts described above and in particular at 600 mg dose can be used. In another example, the DKK1 antibody (e.g., DKNK-0) 1) can be dosed on Day 1 of a 14-day cycle and then repeated on Day 1 for every additional cycle. In a particular embodiment, during a first cycle of multiple 14-day cycles DKN-01 can be administered on both Day 1 of the cycle at about 400 mg and on Day 8 of the cycle at about 400 mg and then once in every 14-day cycle thereafter. In another particular embodiment, during a first cycle of multiple 21-day cycles, DKN-01 can be administered on both Day 1 and Day 15 of the 21-day cycle at about 600 mg and then once in every 21-day cycle thereafter.

Suitable doses per administration for the VEGF or VEGFR inhibitor (e.g., bevacizumab) can be determined based on the recommended dosing known for standard treatment. For example, a suitable dose per administration of bevacizumab is from about 5 mg/kg to about 100 mg/kg intravenously. The administration can be over the necessary period of time to deliver the desired dose in a safe and effective manner. In some instances the time is at least a 30 minute period, a 60 minute period, a 90 minute period, a 2 hour period, a 3 hour period etc. This administration can be repeated every cycle (e.g., once every 14 days of a 14-day cycle). In a particular embodiment, a suitable dose per administration is about 5 mg/kg using an intravenous route.

An effective amount can be achieved in the methods of the invention by coadministering the combination of an initial amount of DKK1 antibody (or a pharmaceutically acceptable salt, hydrate or solvate thereof), an initial amount of VEGF or VEGFR inhibitor (e.g., bevacizumab) and an initial amount of one or more optional chemotherapeutic agents. It is understood that the administration of the amount of chemotherapeutic agents can vary if more than one chemotherapeutic agent is included. In one embodiment, the components of the combination (e.g., a DKK1 antibody, a VEGF or VEGFR inhibitor (e.g., bevacizumab) and one or more optional chemotherapeutic agents) are each administered in a respective effective amount (e.g., each in an amount which would be therapeutically effective if administered alone). In another embodiment, the components of the combination (e.g., a DKK1 antibody, a VEGF or VEGFR inhibitor (e.g., bevacizumab) and one or more optional chemotherapeutic agents) are each administered in an amount which alone does not provide a therapeutic effect (a sub-therapeutic dose). In yet another embodiment, one or two components of the combination can be administered in an effective amount, while the remaining component is administered in a sub-therapeutic dose. For example, the DKK1 antibody can be administered in a sub-therapeutic dose, the VEGF or VEGFR inhibitor (e.g., bevacizumab) can be administered in an effective amount and the one or more therapeutic agents can be administered each at it effective amount. In yet another embodiment, the DKK1 antibody is DKN-01 and is administered at 400 mg, VEGF or VEGFR inhibitor (e.g., bevacizumab) is administered at 5 mg/kg, the one or more optional chemotherapeutic agents are fluorouracil-based chemotherapeutic selected from 5-FU (fluorouracil), FOLFIRI and FOLFOX (e.g., modified FOLFOX6) and are administered as described herein.

In a particular embodiment, the subject will receive DKN-01 (administered IV) on Day 1 of each 14-day cycle, at a dose of 400 mg, with an additional loading dose of 400 mg administered on D8 of C1 only, in combination with either of following two regimens on Day 1 of each 14-day cycle:
FOLFIRI plus bevacizumab: 90-min IV infusion of bevacizumab (5 mg/kg) and a 90-min IV infusion of irinotecan (180 mg/m2) followed by a simplified LV5FU2 regimen (leucovorin [400 mg/m2] and bolus fluorouracil [400 mg/m2] on day 1 and a 46-h infusion of fluorouracil [2400 mg/m2]).
Modified FOLFOX6 (mFOLFOX6) plus bevacizumab: 90-min IV infusion of bevacizumab (5 mg/kg) followed by mFOLFOX6 (Day 1: oxaliplatin 85 mg/m2, folinic acid 400 mg/m2, and fluorouracil 400 mg/m2 IV bolus and then 2,400 mg/m2 over 46 hours continuous infusion).

Additional therapeutic agents, for example, agents used in targeted therapy or immune therapy can be administered to the subject. Such agents include, but are not limited to, PI3K inhibitors and immune checkpoint inhibitors. The additional agents can be administered according to known methods.

As used herein, the term “subject” refers to a mammal, preferably a human, but can also mean an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein “treating” includes achieving, partially or substantially, delaying, inhibiting or preventing the progression of clinical indications related to the colorectal cancer. For example, “treating” includes reduction in tumor growth, or prevention of further growth, as detected by standard imaging methods known in the art, including, for example, computed tomography (CT) scan, magnetic resonance imaging (MRI), chest x-ray, and CT/positron emission tomography (CT/PET) scans, and evaluated according to guidelines and methods known in the art. For example, responses to treatment can be evaluated through the Response Evaluation Criteria in Solid Tumors (RECIST) (Revised RECIST Guideline version 1.1: see Eisenhauer et al., Eur. J. Cancer 45(2): 228-47, 2009). Thus, in some embodiments, “treating” refers to a Complete Response (CR), which is defined according to the RECIST guideline as the disappearance of all target lesions, or a Partial Response (PR), which is defined as at least a 30% decrease in the sum of diameter of target lesions, taking as reference the baseline sum diameters. Other means for evaluating tumor response to treatment include evaluation of tumor markers and evaluation of performance status (e.g., assessment of creatinine clearance; see Cockcroft and Gault, Nephron. 16:31-41, 1976, assessment of carcinoembryonic antigen (CEA) levels in the blood and assessment of Cancer antigen 19-9 (CA 19-9) levels in the blood).

An “objective response” refers to a measurable response, including complete response (CR) or partial response (PR). In some embodiments, the term “objective response rate” (ORR) refers to the sum of complete response (CR) rate and partial response (PR) rate.

“Complete response” or “CR,” as used herein, means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment.

For example, in some embodiments, PR refers to at least a 30% decrease in the sum of the longest diameters (SLD) of target lesions, taking as reference the baseline SLD.

As used herein, “progressive disease” or “PD” refers to at least a 20% increase in the SLD of target lesions, taking as reference the smallest SLD recorded since the treatment started or the presence of one or more new lesions.

### Pharmaceutical Composition

The components of the combination therapy (e.g., the DKK1 antibody, VEGF or VEGFR inhibitor (e.g., bevacizumab) and one or more chemotherapeutic agents) can be incorporated into pharmaceutical compositions suitable for administration. Such compositions typically comprise the DKK1 antibody, or the VEGF or VEGFR inhibitor (e.g., bevaciaumab) and one or more optional chemotherapeutic agents, separate or together in any combination, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL(TM) (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the combination (e.g., a DKK1 antibody, a VEGF or VEGFR inhibitor (e.g., bevacizumab) and one or more optional chemotherapeutic agents) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid-derivatives. Transmucosal administration can be accomplished through the use of nasal sprays or suppositories.

For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As used herein, the term “kRas gene” refers to a human gene having the NCBI Gene ID: 3845.

As used herein, the term “nRas gene” refers to a human gene having the NCBI ID: 4893.

Accordingly, in a first embodiment, the method comprises co-administering to the subject suffering from colorectal cancer a DKK1 antibody, or antigen binding-fragment thereof, a VEGF or VEGFR inhibitor; and optionally one or more chemotherapeutic agents or a pharmaceutically acceptable salt of any of the foregoing, in an effective amount.

In a first aspect of the first embodiment, the VEGF inhibitor is bevacizumab.

In a second aspect of the first embodiment or any particular aspect thereof, the DKK1 antibody is DKN-01.

In a third aspect of the first embodiment, any particular aspect thereof or the first and second aspects thereof the one or more chemotherapeutic agents ia a fluroruracil-based chemotherapeutic selected from: 5-FU (fluorouracil), FOLFIRI, and FOLFOX. In a specific aspect, the FOLFOX is modified FOLFOX6.

In a fourth aspect of the first embodiment, any particular aspect thereof or the first, second or third aspects thereof the method further comprises administering one or more additional therapeutic agents selected from a PI3K inhibitor and/or an immune checkpoint inhibitor. In a particular aspect, the PI3K inhibitor is selected from copanlisib, duvelisib, idelalisib and alpelisib. In a specific aspect, the immune checkpoint inhibitor is a PD-1 inhibitor or a PD-L1 inhibitor. In a particular aspect, the PD-1 inhibitor is selected from nivolumab, pembrolizumab, pidilizumab, AMP-224, sasanlimab, spartalizumab, cemiplimab, retifanlimab, tislelizumab, camrelizumab, budigalimab, zimberelimab, and dostarlimab. In a more particular aspect, the PD-1 inhibitor is pembrolizumab, nivolumab, tislelizumab or budigalimab. In a particular aspect, the PD-L1 inhibitor is selected from atezolizumab, durvalumab, avelumab, envafolimab, BMS-936559, lodapolimab, cosibelimab, sugemalimab and adebrelimab. In a more particular aspect, the PD-L1 inhibitor is atezolizumab.

In a fifth aspect of the first embodiment, any particular aspect thereof or the first, second, third or fourth aspect thereof the subject's colorectal cancer has a detectable level of DKK-1 expression.

In a sixth aspect of the first embodiment, any particular aspect thereof or the first, second, third, fourth or fifth aspect thereof the subject's plasma has a detectable level of DKK1.

In a seventh aspect of the first embodiment, any particular aspect thereof or the first, second, third, fourth, fifth or sixth aspect thereof the subject's serum has a detectable level of DKK1.

In an eighth aspect of the first embodiment, any particular aspect thereof or the first, second, third, fourth, fifth, sixth or seventh aspect thereof the subject has received one prior 5-FU based therapy for colorectal cancer.

In a ninth aspect of the first embodiment, any particular aspect thereof or the first, second, third, fourth, fifth, sixth, seventh or eighth aspect thereof the colorectal cancer is microsatellite stable (MSS).

In tenth aspect of the first embodiment, any particular aspect thereof or the first, second, third, fourth, fifth, sixth, seventh, eighth or ninth aspect thereof the subject's colorectal cancer does not have a BRAF V600E mutation.

In eleventh aspect of the first or second embodiment, any particular aspect thereof or the first, second, third, fourth, fifth, sixth, seventh, eighth, ninth or tenth aspect thereof the colorectal cancer is advanced.

In a twelfth aspect aspect of the first embodiment, any particular aspect thereof or the first, second, third, fourth, fifth, sixth, seventh, eighth, ninth, tenth or eleventh aspect thereof the colorectal cancer is metastatic.

In a thirteenth aspect aspect of the first embodiment, any particular aspect thereof or the first, second, third, fourth, fifth, sixth, seventh, eighth, ninth, tenth, eleventh or twelfth aspect thereof the colorectal cancer is an adenocarcinoma.

In a fourteenth aspect of the first embodiment, any particular aspect thereof or the first, second, third, fourth, fifth sixth, seventh, eighth, ninth, tenth, eleventh, twelfth or thirteenth aspect thereof the treatment is administered in the course of one or more 14-day cycles.

In a fifteenth aspect of the first embodiment, any particular aspect thereof or the first, second, third, fourth, fifth sixth, seventh, eighth, ninth, tenth, eleventh, twelfth, thirteenth or fourteenth aspect thereof the DKK1 antibody, or antigen binding-fragment thereof is administered on day 1 of a 14-day cycle at an amount of 400 mg.

In a sixteenth aspect of the first embodiment, any particular aspect thereof or the first, second, third, fourth, fifth sixth, seventh, eighth, ninth, tenth, eleventh, twelfth, thirteenth, fourteenth or fifteenth aspect thereof the VEGF inhibitor is bevacizumab and is administered at 5 mg/kg on day 1 of a 14-day cycle.

In a seventeenth aspect of the first embodiment, any particular aspect thereof or the first, second, third, fourth, fifth, sixth, seventh, eighth, ninth, tenth, eleventh, twelfth, thirteenth, fourteenth, fifteenth or sixteenth aspect thereof the DKK1 antibody, or antigen binding-fragment thereof, comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO: 1, LCDR2 has the amino sequence of SEQ ID NO:2, LCDR3 has the amino sequence of SEQ ID NO: 3, HCDR1 has the amino sequence of SEQ ID NO:4, HCDR2 has the amino sequence of SEQ ID NO:5, and an HCDR3 has the amino sequence of SEQ ID NO:6. In a particular aspect of the seventeenth aspect, the LCVR comprises the amino acid sequence of SEQ ID NO: 7 and the HCVR comprises the amino acid sequence of SEQ ID NO: 8. In a further particular aspect, the LCVR and HCVR comprise amino acid sequences selected from the group consisting of: (i) a LCVR comprising the amino acid sequence of SEQ ID NO: 9 and a HCVR comprising the amino acid sequence of SEQ ID NO: 10; (ii) a LCVR comprising the amino acid sequence of SEQ ID NO: 11 and a HCVR comprising the amino acid sequence of SEQ ID NO: 12; (iii) a LCVR comprising the amino acid sequence of SEQ ID NO: 13 and a HCVR comprising the amino acid sequence of SEQ ID NO: 10; (iv) a LCVR comprising the amino acid sequence of SEQ ID NO: 14 and a HCVR comprising the amino acid sequence of SEQ ID NO: 10. In yet another particular aspect of the seventeenth aspect, the LCVR comprises the amino acid sequence of SEQ ID NO: 11 and the HCVR comprises the amino acid sequence of SEQ ID NO: 12.

In a eighteenth aspect of the first or second embodiment, any particular aspect thereof or the first, second, third, fourth, fifth, sixth, seventh, eighth, ninth, tenth, eleventh, twelfth, thirteenth, fourteenth, fifteenth, sixteenth or seventeenth aspect thereof, the DKK1 antibody comprises a heavy chain and a light chain amino acid sequence selected from the group consisting of a) a heavy chain comprising the amino acid sequence of SEQ ID NO: 19 and light chain comprising the amino acid sequence of SEQ ID NO: 16, b) a heavy chain comprising the amino acid sequence of SEQ ID NO: 17 and a light chain comprising the amino acid sequence of SEQ ID NO: 18, c) a heavy chain comprising the amino acid sequence of SEQ ID NO: 19 and a light chain comprising the amino acid sequence of SEQ ID NO: 20, and d) a heavy chain comprising the amino acid sequence of SEQ ID NO: 19 and a light chain comprising the amino acid sequence of SEQ ID NO: 21.

In a nineteenth aspect of the first embodiment, any particular aspect thereof or the first, second, third, fourth, fifth, sixth, seventh, eighth, ninth, tenth, eleventh, twelfth, thirteenth, fourteenth, fifteenth, sixteenth, or seventeenth aspect thereof the DKK1 antibody comprises a heavy chain comprising the amino acid sequence of SEQ ID NO: 17 and a light chain comprising the amino acid sequence of SEQ ID NO: 18.

In a twentieth aspect of the first embodiment, any particular aspect thereof or the first, second, third, fourth, fifth, sixth, seventh, eighth, ninth, tenth, eleventh, twelfth, thirteenth, fourteenth, fifteenth, sixteenth, seventeenth, eighteenth or nineteenth aspect thereof the DKK1 antibody is DKN-01.

In a 21^{st} aspect of the 1^{st} embodiment, or any aspect thereof, the subject is a human.

In a 22^{nd} aspect of the 1^{st} embodiment, or any aspect thereof, the subject is a rapid progressor.

In a 23^{rd} aspect of the 1^{st} embodiment, or any aspect thereof, the subject harbors a mutation in a kRas gene or an nRas gene.

In a 24^{th} aspect of 1^{st} embodiment, or any aspect thereof, the subject suffers from liver metastases.

In a 25^{th} aspect of the 1^{st} embodiment, or any aspects thereof, the colorectal cancer is a rectal cancer.

In a second embodiment, the invention relates to a pharmaceutical composition comprising: a DKK1 antibody, or antigen binding-fragment thereof; bevacizumab; and optionally one or more chemotherapeutic agents or a pharmaceutically acceptable salt of any of the foregoing.

In various aspects, the 2^{nd} embodiment is described above with respect to the 1^{st} through 25^{th} aspect of the 1^{st} embodiment.

In a 3^{rd} embodiment, the present invention is a pharmaceutical composition comprising: a DKK1 antibody, or antigen binding-fragment thereof; a VEGF or a VEGFR inhibitor; and one or more chemotherapeutic agents or a pharmaceutically acceptable salt of any of the foregoing.

In various aspects, the 3^{rd} embodiment is described above with respect to the 1^{st} through 25^{th} aspect of the 1^{st} embodiment.

In a 4^{th} embodiment, the present invention is a kit comprising a DKK1 antibody, or antigen binding-fragment thereof; a VEGF or a VEGFR inhibitor; one or more chemotherapeutic agents or a pharmaceutically acceptable salt thereof; and instructions for use.

In various aspects, the 3^{rd} embodiment is described above with respect to the 1st through 25^{th} aspect of the 1^{st} embodiment.

In a 5^{th} embodiment, the present invention is a comprising a DKK1 antibody, or antigen binding-fragment thereof; bevacizumab; one or more chemotherapeutic agents or a pharmaceutically acceptable salt thereof; and instructions for use.

In various aspects, the 5^{th} embodiment is described above with respect to the 1st through 25^{th} aspect of the 1^{st} embodiment.

### EXEMPLIFICATION

### Example I: Clinical Protocol

Described is a Phase 2 randomized, open-label, two-part, multicenter study with a safety run-in to evaluate efficacy and safety of DKN-01 plus [FOLFIRI or FOLFOX and bevacizumab] versus standard of care (SOC) [FOLFIRI or FOLFOX and bevacizumab] as second-line treatment of advanced CRC patients. (See NCT05480306 at clincaltrials.gov)

In Parts A and B, approximately 150 evaluable adult advanced CRC patients with measurable disease (RECIST v1.1) who have radiographically progressed during or following 1 line of systemic treatment will be enrolled in the study.

The study consists of a Screening Period, a Treatment Period, a Safety Follow-up Period (SFUP) and a Long-Term Follow-up Period (LTFU). Patients will be followed in the SFUP for approximately 30 days (+7 days) after the last administration of study drug and then enter the LTFU period to be followed for survival and subsequent therapies. Additionally, patients that ended study treatment for a reason unrelated to progressive disease [PD] will also be followed for disease progression in the LTFU period.

### Part A: Safety Run-In

Enough patients will be enrolled in Part A to ensure that at least 20 patients are evaluable for review by the Safety Review Committee (SRC). Evaluable patients are defined as those completing all treatment doses in C1 and C2 (3 DKN-01 doses total) and completing all the safety evaluations through the end of C2; dose delays permitted. Non-evaluable patients may be replaced.

Subjects determined to be eligible will be registered (ie, enrolled) in Part A. Registration can occur up to 3 days prior to CID1.

Part A patients will receive DKN-01 (also known as LY2812176) administered intravenously (IV) on Day (D) 1 of each 14-day cycle (30-min infusion), at a dose of 400 mg, with an additional loading dose of 400 mg administered on D8 of Cycle (C) 1 only, in combination with either of following regimens also on D1, at the Investigator's choice, as suggested below or per institutional standard practice:
FOLFIRI plus bevacizumab: 90-min IV infusion of bevacizumab (5 mg/kg) followed by a 90-min IV infusion of irinotecan (180 mg/m2) followed by a simplified LV5FU2 regimen (leucovorin [400 mg/m2] and bolus fluorouracil [400 mg/m2] on D1 and a 46-h infusion of fluorouracil [2400 mg/m2]).
Modified FOLFOX6 (mFOLFOX6) plus bevacizumab: 90-min IV infusion of bevacizumab (5 mg/kg) followed by mFOLFOX6 (D1: oxaliplatin 85 mg/m2, folinic acid 400 mg/m2, and fluorouracil 400 mg/m2 IV bolus and then 2,400 mg/m2 of lurorouracil over 46 hours continuous infusion).

Treatment continues in repeating 14-day cycles until patient meets criteria for discontinuation or is no longer deriving clinical benefit.

After all the evaluable patients are monitored for two cycles (a minimum of 28 days), the Safety Review Committee (SRC) will review the overall safety profile of the Part A patients and determine if the 400 mg DKN-01 dose plus FOLFIRI/FOLFOX plus bevacizumab is safe and tolerable to start Part B.

### Part B

Approximately 130 patients will be randomized 1:1 to either the experimental or control arm. Randomization may occur up to 3 days prior to C1D1.

Experimental arm patients receive DKN-01 administered IV on Day 1 of each 14-day cycle, at a dose of 400 mg, with an additional loading dose of 400 mg administered on D8 of C1 only, in combination with either of following two regimens on D1 of each cycle, at the Investigator's choice, as suggested below or per institutional standard practice:
FOLFIRI plus bevacizumab: 90-min IV infusion of bevacizumab (5 mg/kg) followed by a 90-min IV infusion of irinotecan (180 mg/m2) followed by a simplified LV5FU2 regimen (leucovorin [400 mg/m2] and bolus fluorouracil [400 mg/m2] on day 1 and a 46-h infusion of fluorouracil [2400 mg/m2]); or
Modified FOLFOX6 (mFOLFOX6) plus bevacizumab: 90-min IV infusion of bevacizumab (5 mg/kg) followed by a 2-hour infusion of mFOLFOX6 (Day 1: oxaliplatin 85 mg/m2, folinic acid 400 mg/m2, and fluorouracil 400 mg/m2 IV bolus and then 2,400 mg/m2 over 46 hours continuous infusion).

Control arm patients receive only Standard of Care (SOC) being FOLFIRI or mFOLFOX6 (at the Investigator's choice) plus bevacizumab, with this SOC regimen administered as described above for the experimental arm.

Treatment continues in repeating 14-day cycles until patient meets criteria for discontinuation or is no longer deriving clinical benefit.

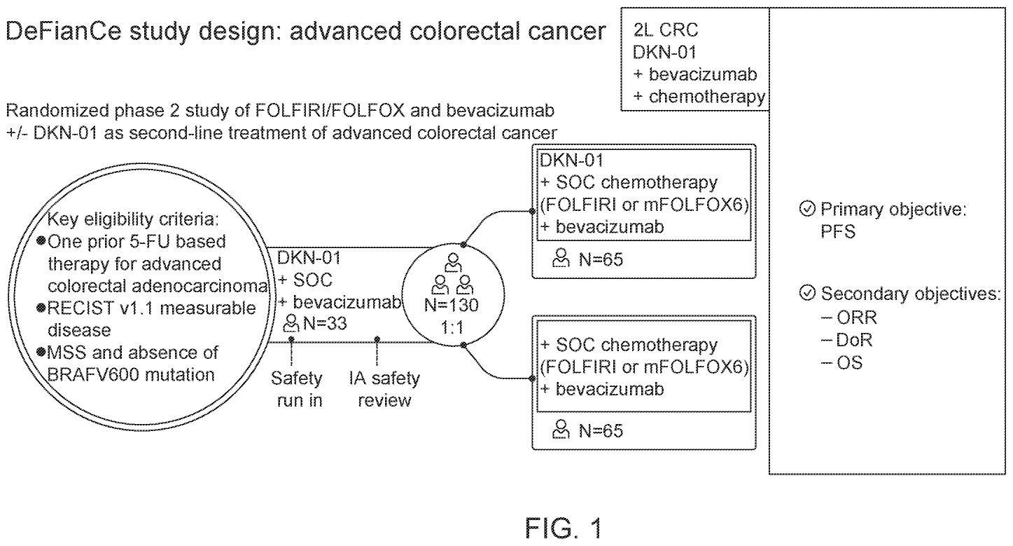

The study design schematic is presented in FIG. 1

### Endpoint Definitions

### Time-to-Event and Duration Endpoints

Progression-free survival (PFS) is a primary outcome measure and is defined for each patient as the interval from the date of randomization (or date of registration for Part A patients) until radiological assessed tumor progression or death from any cause, whichever occurs first. Patients who discontinue from the study for other reasons than disease progression will be censored at the time of the last radiological scans. Patients who start an alternative anti-cancer therapy (with the exception of palliative radiation therapy to a pre-existing, non-target lesion without PD) will be treated as censored at that time. Patients lost to follow-up will be censored at the time of the last known contact. PFS is determined by the investigator per RECIST v1.1 of DKN-01 plus SOC versus SOC alone.

Overall Survival (OS) is defined for each patient as the time from the date of randomization (or date of registration for Part A patients) to death due to any cause. If the patient is alive or lost to follow-up at the time of data analysis, OS data will be censored on the last date the patient is known to be alive or the data cut-off date, whichever is earlier.

Duration of response (DoR) is defined only for responders (patients with a BOR of CR or PR) as the time from initial response (CR or PR) until radiographically documented progressive disease or death due to any cause, whichever is earlier. Patients who do not experience PD or death at the time of the analysis will be censored using the same rules as described for PFS.

Duration of complete response (DoCR) is defined as the time from initial CR until radiographically documented progressive disease or death due to any cause, whichever occurs first. Patients who do not experience PD or death at the time of the analysis will be censored using the same rules as described for PFS.

Duration of clinical benefit (DoCB) is defined as the time from the date of randomization (or date of registration for Part A patients) to the time of progressive disease or death due to any cause, whichever occurs first. Patients who do not experience PD or death at the time of the analysis will be censored using the same rules as described for PFS.

Time to best response (TTRBest), for patients with a BOR of CR or PR, is defined as the time from the date of randomization (or date of registration for Part A patients) to the assessment date of the first BOR of either CR or PR.

Time to first response (TTRFirst) is defined as the time from the date of randomization (or date of registration for Part A patients) to the assessment date of the first instance of an overall response of CR or PR.

Time to response (TTR) is defined as the time from the date of randomization (or dates of registration for Part A patients) to the assessment date of the first instance of an overall response of Complete Response (CR) or Partial Response (PR).

Best Overall Response (BOR) is defined as the best response recorded for a patient from the start of a study drug treatment until the end of treatment taking into account any requirement for confirmation.

### Response Rate Endpoints

Objective response rate (ORR) is defined as the proportion of patients achieving a best overall response (BOR) of complete response (CR) or partial response (PR) as assessed by the Investigator per RECIST v1.1. Responses evaluated after switching to another anti-cancer therapy will be excluded.

Durable Clinical Benefit (DCB) rate is defined as the proportion of patients presenting a duration of clinical benefit (DoCB) for ≥180 days from randomization (or date of registration for Part A patients). Patients who have a best overall response (BOR) of PD or those having clinical benefit but DoCB lasting <180 days will be considered as ‘non-DCB.

Disease control rate (DCR) is defined as the proportion of patients presenting with a best overall response (BOR) of complete response, partial response, or stable disease for a duration of at least 8 weeks from randomization (or date of registration for Part A patients) as assessed by the Investigator using RECIST v1.1.

### Analysis of the Primary Efficacy Endpoint

The primary efficacy analysis will be the comparison of investigator assessed PFS between the two treatment arms in the ITT population, using the one-sided stratified log-rank test. In other words PFS, as determined by the Investigator per RECIST v1. 1 of DKN-01 plus SOC versus SOC.

Data will be summarized in Kaplan-Meier curves together with medians and 95% confidence intervals for those medians. The Cox-Proportional Hazards model, stratified for the randomization factors, will be used to obtain a hazard ratio together with its 95% confidence interval.

### Analysis of Secondary Efficacy Endpoints

ORR will be compared between the two treatment arms in Part B in the ITT population based upon the investigator assessment using the Cochran-Mantel-Haenzel test as for the analysis of PFS. Results will be reported in terms of an odds ratio and associated 95% confidence interval. In other words, ORR, as determined by the Investigator per RECIST v1.1 of DKN-01 plus SOC versus SOC.

OS will be compared between the two treatment arms in Part B in the ITT population using the same methods of analysis as for PFS. In other words, OS with DKN-01 plus SOC versus SOC.

There will be no formal statistical testing for DoR, as this is not a randomized comparison, and these data will be evaluated through the presentation of descriptive statistics, include Kaplan-Meier curves. In other words, DoR, as determined by the Investigator per RECIST v1.1 of DKN-01 plus SOC versus SOC.

Tumor response-based endpoints, including ORR, DOR, PFS, time to events, will be analyzed based on investigator-reviewed results according to RECIST 1.1. The independent data review will provide RECIST measurements for each visit for each patient. Central review data, if done, will be considered exploratory. Results of this independent review will not be communicated to the Investigators during the study. The management of patients will be based solely upon the results of the RECIST 1.1 assessment conducted by the Investigator.

### Inclusion Criteria:

Histologically proven diagnosis of advanced colorectal adenocarcinoma (by local laboratory and local clinical guidelines) with documented objective radiographic or symptomatic disease progression following first-line systemic therapy with any fluoropyrimidine-based regimen for advanced disease (except FOLFOXIRI, see list of Exclusion Criteria).
a) Patients may have received prior neoadjuvant or adjuvant therapy which could have included irinotecan or oxaliplatin. If progression has occurred within 6 months from last dose of neoadjuvant or adjuvant treatment, this regimen will be considered as the one line of systemic therapy for advanced disease.
   i. If assigned to receive FOLFIRI, patient must not have received irinotecan as part of first-line systemic therapy
   ii. If assigned to receive FOLFOX, patient must not have received oxaliplatin as part of first line systemic therapy.
   iii. Prior treatment with an anti-VEGF or anti-EGFR therapy is allowed as first-line and/or maintenance systemic therapy.
b) Presence of at least one measurable lesion assessed by CT and/or MRI according to RECIST 1.1. (A lesion in an area subjected to prior loco-regional therapy, including previous radiotherapy, is not considered measurable unless there has been demonstrated progression in the lesion since the therapy as defined by RECIST v1.1.)

### Exclusion Criteria

The following is a list of exclusion criteria to be used in the study. The list of exclusion criteria is exemplary and other criteria may be used during the study.

Microsatellite instability-high (MSI-H)/mismatch repair-deficient (dMMR) and/or BRAF V600E mutation positive colorectal cancer. Prior therapy with an anti-DKK1 agent. Prior therapy with FOLFOXIRI. Prior therapy with an anti-programmed cell death protein ligand-1 [PD-(L) 1] or anti-programmed cell death protein ligand-2 (PD-L2) or any other antibody or drug specifically targeting T-cell co-stimulation or coinhibitory checkpoint pathways in any treatment setting (including adjuvant/neoadjuvant). Systemic anti-cancer therapy within 28 days prior to first dose of study drug. Major surgery within 28 days prior to first dose of study drug. Treatment with radiation therapy within 14 days prior to first dose of study drug.

Enrollment in Part A of this study has been completed, treatment and follow up are ongoing. The Experimental Arm of the protocol described above was followed with respect to 33 patients. The results are described below in Example II.

### Example II: Preliminary Results of Part a of the DeFianCe Study

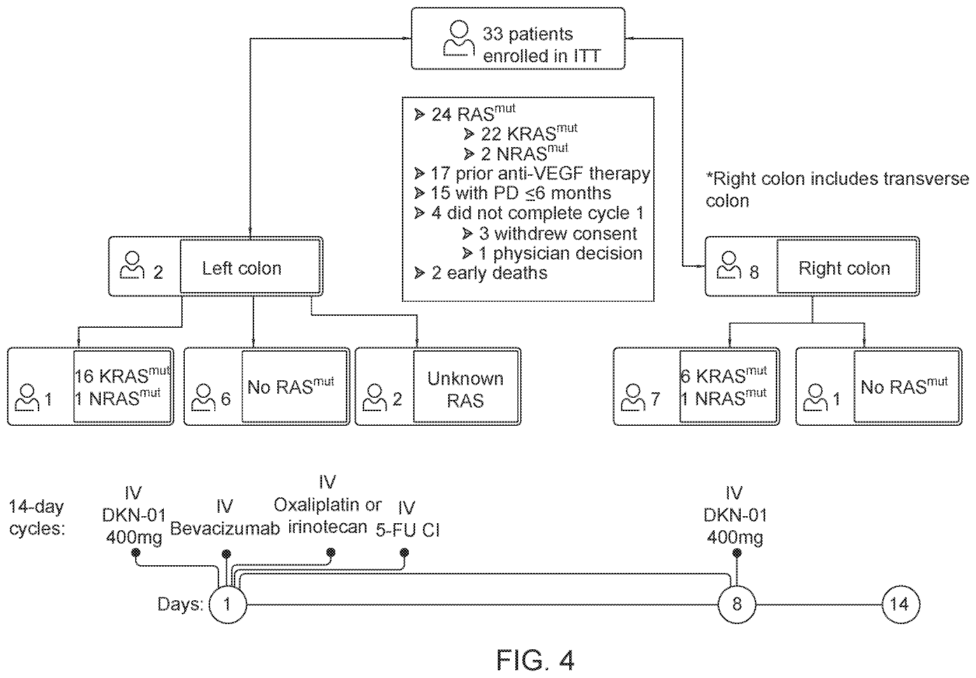

In Part A of the DeFianCe study, 33 colorectal cancer (CRC) patients were treated according to the Part A protocol described above. FIG. 4 shows a consort diagram of Part A of the DeFianCe Study. The results described herein are based on preliminary data from Part A of the study and the final data is not yet available.

The patient population in Part A was characterized as follows:
76% left colon (primary tumor location)
52% prior bevacizumab treatment
68% KRAS mutations
70% with liver metastasis
45% 1L PD≤6 months

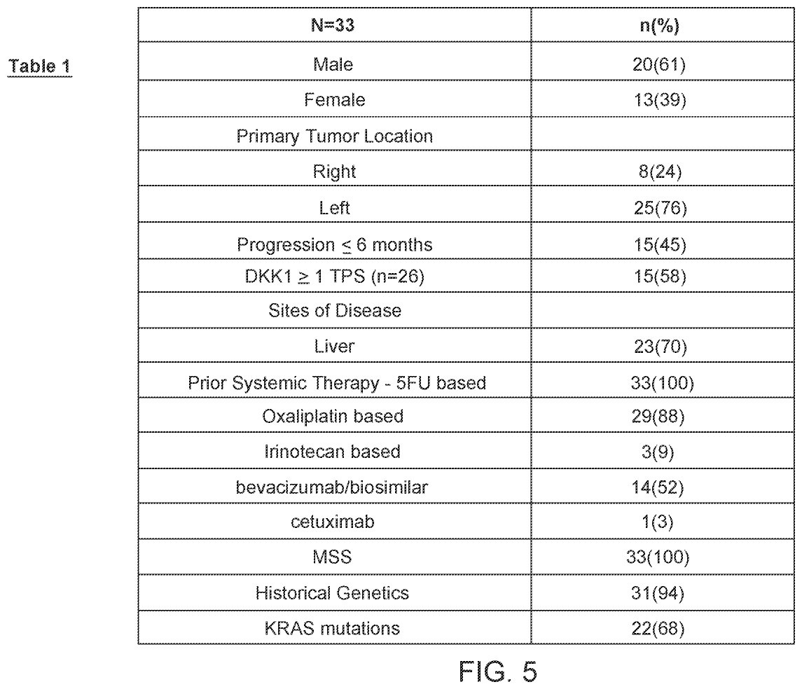

Further characterization of the patients is found in Table 1, shown in FIG. 5.

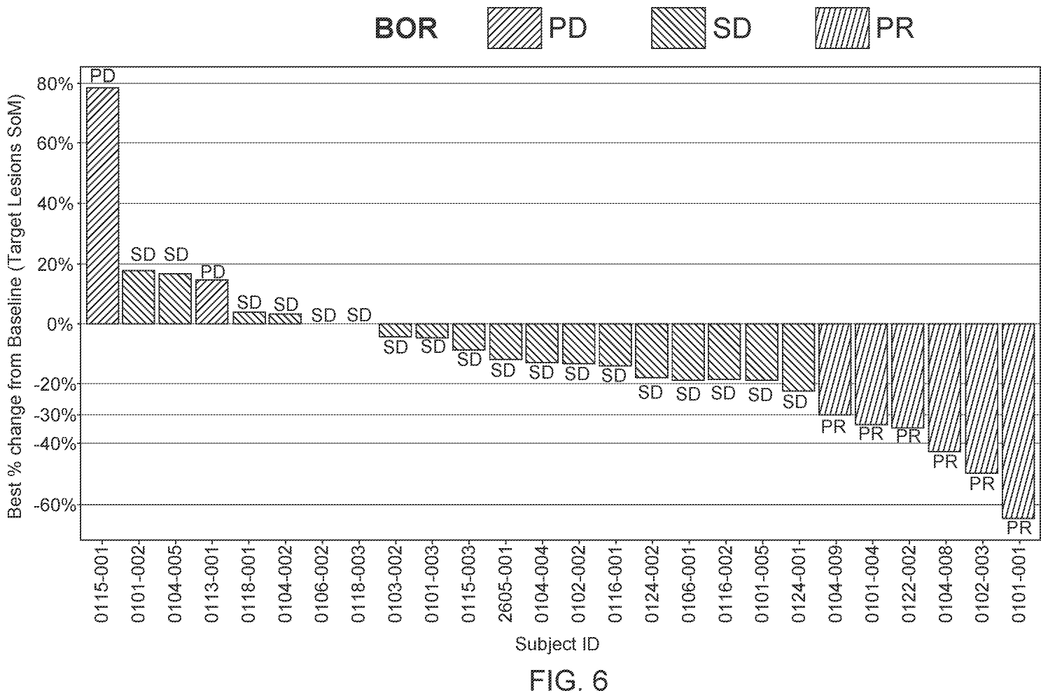

It was determined that 26 of the 33 patients were response evaluable (RE). The treatment regime shown in FIG. 4 was evaluated by Best Overall Response (BOR, best % change from baseline (target lesion, Sum of Measurement (SOM)). The results are shown in FIG. 6 (the results are preliminary data from Part A of the study and the final data is not yet available). It can be seen that the Objective Response Rate (ORR) in RE patients, at the time of the preliminary data assessment, was 6/26=23%, and Disease Control Rate (DCR) in RE patients was 24/26=92%. The results are summarized in Table 2.

TABLE 2

| | Objective | Disease | Partial | Stable | Progressive |
|---|---|---|---|---|---|
| | Response | Control | Response | Disease | Disease |
| | Rate (%) | Rate (%) | n (%) | n (%) | n (%) |
| Overall, | 23 | 92 | 6 (23) | 18 (69) | 2 (8) |
| n = 26 | | | | | |

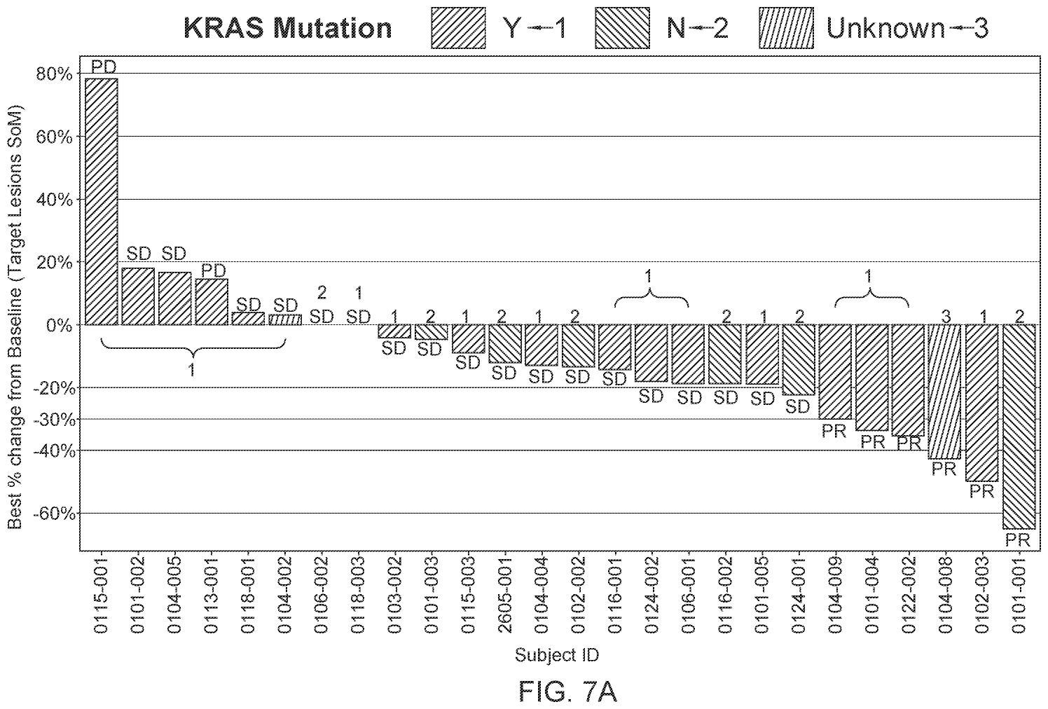

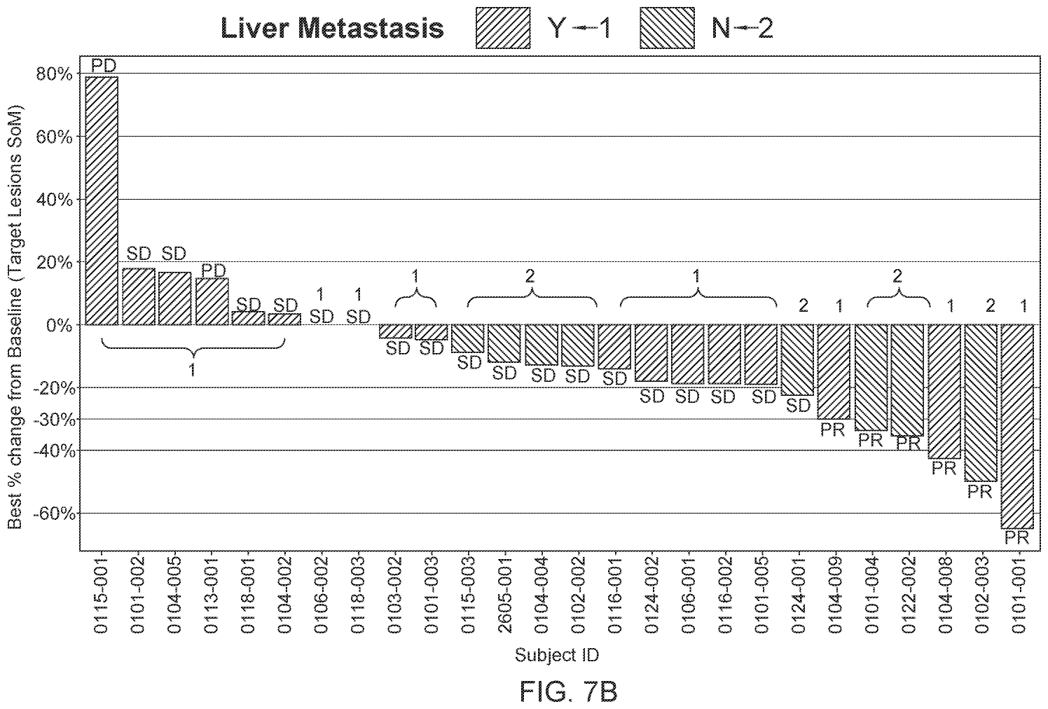

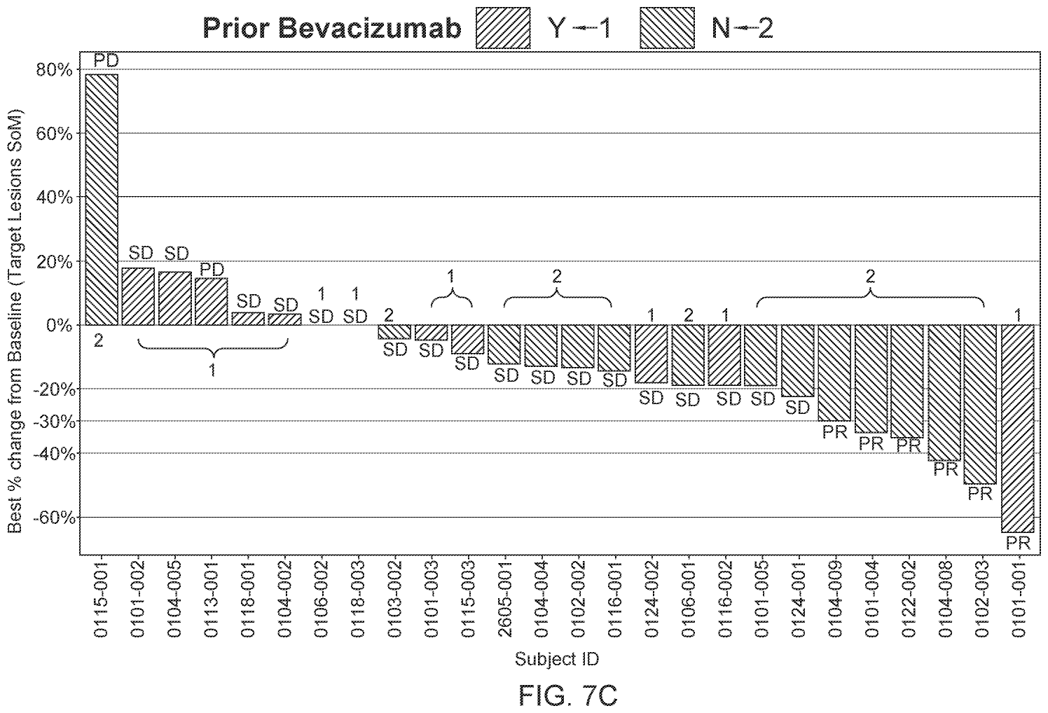

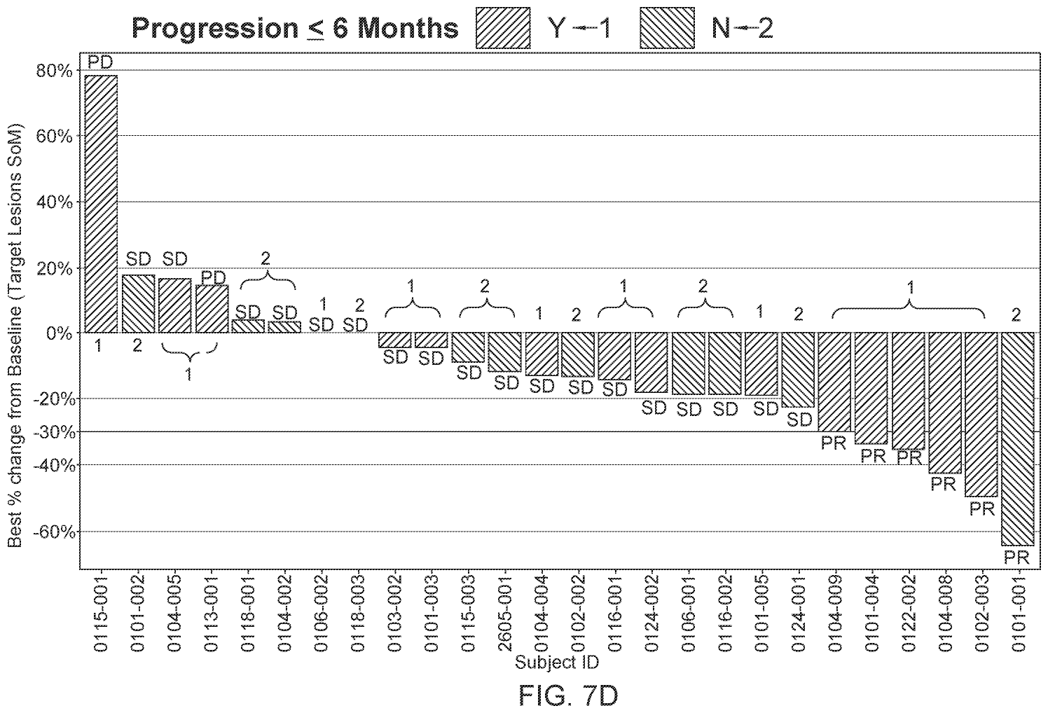

The 26 response-evaluable patients were also categorized by the presence of a KRAS mutation. The results are presented in FIG. 7A and summarized in Table 3. As can be seen, the majority of the patients harboring KRAS mutations (patients expected to have poorer outcomes than those harboring KRAS wild type tumors) demonstrated either a PR or SD).

TABLE 3

| | Objective | Disease | Partial | Stable | Progressive |
|---|---|---|---|---|---|
| | Response | Control | Response | Disease | Disease |
| | Rate (%) | Rate (%) | n (%) | n (%) | n (%) |
| Overall, | 23 | 92 | 6 (23) | 18 (69) | 2 (8) |
| n = 26 | | | | | |
| KRAS^{mut} | 24 | 88 | 4 (24) | 11 (65) | 2 (12) |
| n = 17 | | | | | |

The 26 response-evaluable patients were also categorized by the presence of liver metastases. The results are presented in FIG. 7B and summarized in Table 4. As can be seen, the majority of the patients having liver metastases (patients expected to have a poor prognosis) demonstrated either PR or SD.

TABLE 4

| | Objective | Disease | Partial | Stable | Progressive |
|---|---|---|---|---|---|
| | Response | Control | Response | Disease | Disease |
| | Rate (%) | Rate (%) | n (%) | n (%) | n (%) |
| Overall, | 23 | 92 | 6 (23) | 18 69) | 2 (8) |
| n = 26 | | | | | |
| Liver | 18 | 88 | 3 (18) | 13 (89) | 2 (12) |
| mets, | | | | | |
| n = 18 | | | | | |

The 26 response-evaluable patients were also categorized by whether the patient had the undergone a prior Bevacizumab (anti-VEGF) treatment. The results are presented in FIG. 7C.

Each of the 26 response-evaluable patients was also assessed to determine if the patient would be identified as a “rapid progressor.” As used herein, the phrase “rapid progressor” refers to a patient who shows evidence of progressive disease (PD) on or within 6 months after the last dose of one prior line of systemic anti-cancer therapy (e.g., 5-FU+/−oxaliplatin or irinotecan) administered for metastatic disease. The results are presented in FIG. 7D.

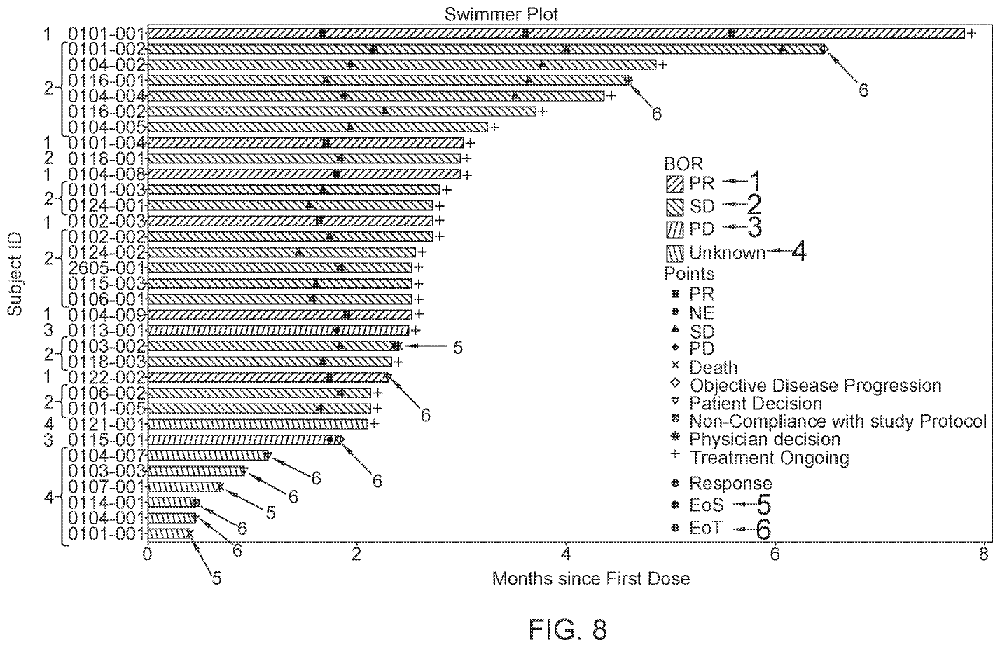

Preliminary data shows that 22 of the 33 patients remained on study therapy at the time of data assessment. As can be seen in FIG. 8, the majority of the patients who remained on study demonstrated either stable disease (SD identified with a number 2 in FIG. 8) or partial response (PR identified with a number 1 in FIG. 8). In FIG. 8, the categories of “Response” are: “EoS”, referring to end of study and “EoT”, referring to end of treatment.

### Example III: Combination of PIK3CA (Phosphatidylinositol-4,5-Bisphosphate 3-Kinase Catalytic Subunit Alpha) Inhibitor BYL719 and DKN-01

A study was initiated to evaluate growth of a human colorectal cancer cell line, HCT116, with and without the PI3KCA H1047R mutation in mice treated with DKN-01, the PI3KCA inhibitor BYL719 (Alpelisib), or the combination. Female BALB/c scid mice were inoculated subcutaneously with HCT116 human colon carcinoma cells with or without the H1047R mutation on Day 0. On Day 17, when tumors volumes had reached 50-75 mm3, animals were randomized into treatment groups and dosing was initiated.

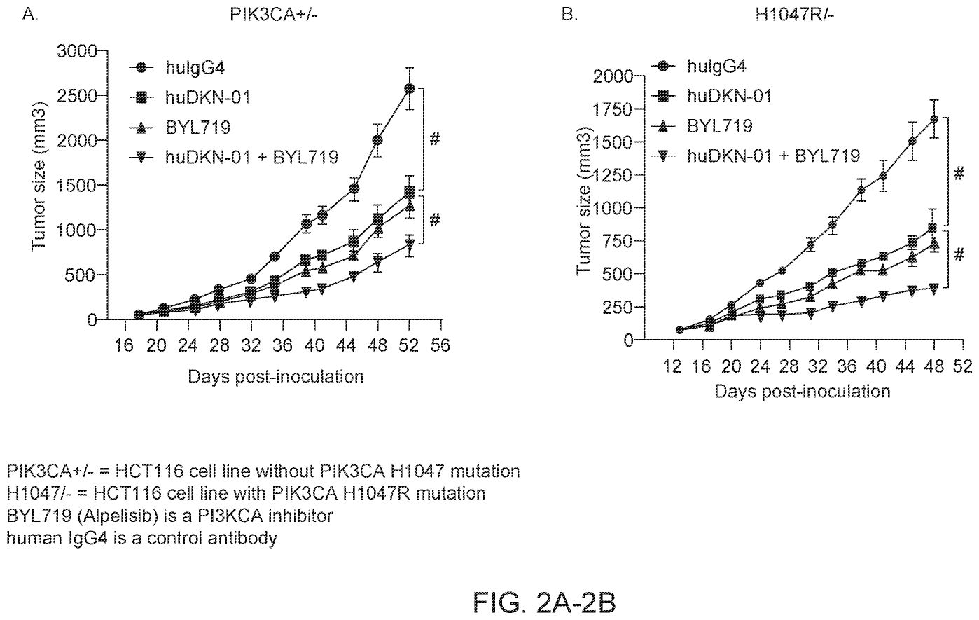

In the HCT116 PIK3CA+/− inoculated mice (HCT116 cell line without the PIK3CA H1047 mutation), DKN-01 and BYL719 monotherapy resulted in Tumor Growth Inhibition (TGI) of 45% (p=0.03) and 50% (p=0.005), respectively at Day 52 (Treatment Day 36) compared to the IgG4 control. The combination therapy resulted in an overall TGI of 68% (p<0.0001) at Day 52 when compared to the IgG4 control. Additionally, the combination therapy when compared to DKN-01 and BYL719 monotherapy resulted in TGIs of 42% (p=0.02) and 36% (p=0.04), respectively (FIG. 2A).

In the HCT116 H1047/− (HCT116 cell line with H1047R mutation inoculated mice), DKN-01 and BYL719 monotherapy resulted in TGIs of 49% (p=0.03) and 56% (p=0.001), respectively at Day 48 (Treatment Day 36) compared to the IgG4 control. The combination therapy resulted in an overall TGI of 77% (p<0.0001) at Day 48 when compared to the IgG4 control. Additionally, the combination therapy when compared to mDKN-0) 1 and BYL719 monotherapy resulted in TGIs of 54% (p=0.03) and 48% (p=0.01) (FIG. 2B).

A detailed protocol for the model used is as follows:

Female BALB/c scid mice (10 per group) were inoculated subcutaneously in the right flank with 10×10\hat{ }6 HCT116 human colorectal carcinoma cells on Day 0. Human IgG4 control (10 mg/kg) and DKN-01 (5 mg/kg) were administered intraperitoneally (IP) twice weekly. BYL719 (25 mg/kg) was administered orally (PO) every day (QD) for the duration of the study. Tumor measurements were performed every 3-4 days with calipers and volume (V) was calculated using the following formula: V=½(length [mm]×width [mm]\hat{ }2). Data are shown as mean tumor volumes (MTV)+SEM. #, p<0.05. Percent TGI =[(MTV Control)−(MTV Treated)]/(MTV Control). Comparisons between groups at study termination were performed using a one-way repeated measures ANOVA with Tukey's multiple comparisons test.

### Example IV: DKN-01 Activity in Combination with Anti-PD-1 Antibody in a Colorectal Cancer Model

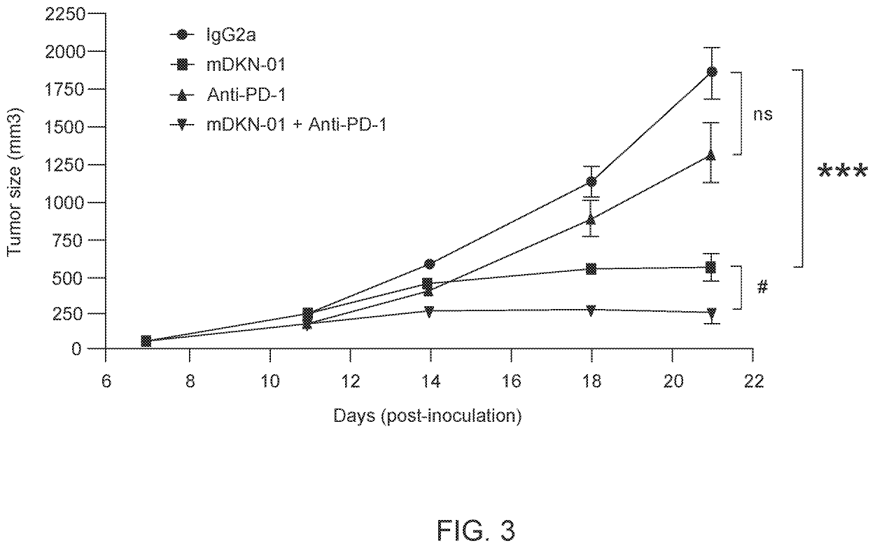

The activity of mDKN-01 and an anti-PD-1 antibody alone and in combination was assessed in a CT26 syngeneic model (FIG. 3). Female BALB/c mice were inoculated subcutaneously with CT26 mouse colon carcinoma cells on Day 0. On Day 7, when tumors volumes had reached 50-65 mm3, animals were randomized into treatment groups and dosing was initiated. mDKN-01 and anti-PD-1 monotherapy resulted in Tumor Growth Inhibition (TGI) of 71% (p<0.0001) and 29% (p=0.20), respectively at Day 21 (Treatment Day 14) compared to the IgG2a control. The combination therapy resulted in an overall TGI of 88% (p<0.0001) at Day 21 when compared to the IgG2a control. Additionally, the combination therapy when compared to mDKN-01 and anti-PD-1 monotherapy resulted in TGIs of 58% (p=0.03) and 83% (p=0.0004), respectively indicating that additive efficacy was observed.

A detailed protocol for the model used is as follows:

Female BALB/c mice (15 per group) were inoculated subcutaneously in the right flank with 0.5×10\hat{ }6 CT26 mouse colon carcinoma cells on Day 0. The IgG2a control (10 mg/kg) and a murine surrogate IgG2a DKN-01 (mDKN-01) antibody with a D265A mutation to reduce effector function (10 mg/kg) was administered intraperitoneally (IP) twice weekly. The anti-PD-1 (10 mg/kg) was administered IP every four days (Q4D). Tumor measurements were performed every 3-4 days with calipers and volume (V) was calculated using the following formula: V=½ (length [mm]×width [mm]\hat{ }2). Data are shown as mean tumor volumes (MTV)±SEM. #, p<0.05: ***, p<0.0001. Percent TGI=[(MTV Control)−(MTV Treated)]/(MTV Control). Comparisons between groups were performed using a two-way repeated measures ANOVA with Tukey-Kramer post-hoc tests.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating colorectal cancer in a subject in need of treatment, the method comprising co-administering to the subject:
a) a DKK1 antibody, or antigen binding-fragment thereof;
b) a VEGF or VEGFR inhibitor; and
c) optionally one or more chemotherapeutic agents
or a pharmaceutically acceptable salt of any of the foregoing, in an effective amount.

2. The method of claim 1, wherein the VEGF or VEGFR inhibitor is selected from pazopanib, sunitinib, sorafenib, regorafenib, cabozantinib, lenvatinib, ponatinibcabozantinib, ziv-aflibercept, axitinibtivozanib, ramucirumab, vandetanib, or bevacizumab.

3. The method of claim 1, wherein the VEGF or VEGFR inhibitor is bevacizumab or a biosimilar of bevacizumab.

4. The method of claim 3, wherein the VEGF or VEGFR inhibitor is a biosimilar of bevacizumab selected from mvasi, zirabev, alymsys and vegzelma.

5. The method of any one of claims 1-4, wherein the DKK1 antibody is DKN-01.

6. The method of any one of claims 1-5, wherein the one or more chemotherapeutic agents is a fluorouracil-based chemotherapeutic selected from: 5-FU (fluorouracil), FOLFIRI, FOLFOX.

7. The method of claim 6, wherein the FOLFOX is modified FOLFOX6.

8. The method of any one of claims 1-7, further comprising administration of one or more additional therapeutic agents selected from a PI3K inhibitor and/or an immune checkpoint inhibitor.

9. The method of claim 8, wherein the immune checkpoint inhibitor is PD-1 inhibitor or a PD-L1 inhibitor.

10. The method of claim 9, wherein the PD-1 inhibitor is selected from nivolumab, pembrolizumab, pidilizumab, AMP-224, sasanlimab, spartalizumab, cemiplimab, retifanlimab, tislelizumab, camrelizumab, budigalimab, zimberelimab, and dostarlimab.

11. The method of claim 10, wherein the PD-1 inhibitor is pembrolizumab, nivolumab, tislelizumab, or budigalimab.

12. The method of claim 9, wherein the PD-L1 inhibitor is selected from atezolizumab, durvalumab, avelumab, envafolimab, BMS-936559, lodapolimab, cosibelimab, sugemalimab and adebrelimab.

13. The method of claim 8, wherein the PI3KCA inhibitor is selected from copanlisib, duvelisib, idelalisib and alpelisib.

14. The method of any one of claims 1-13, wherein the subject's colorectal cancer is determined to have a detectable level of tumoral DKK-1 expression.

15. The method of any one of claims 1-13, wherein the subject's plasma has a detectable level of DKK1.

16. The method of any one of claims 1-13, wherein the subject's serum has a detectable level of DKK1.

17. The method of any one of claims 1-16, wherein the subject has received one prior 5-FU based therapy for the colorectal cancer.

18. The method of any one of claims 1-17, wherein the subject's colorectal cancer is microsatellite stable (MSS).

19. The method of any one of claims 1-17, wherein the subject's colorectal cancer does not have a BRAF V600E mutation.

20. The method of any one of claims 1-19, wherein the subject's colorectal cancer is advanced colorectal cancer.

21. The method of any one of claims 1-20, wherein the subject's colorectal cancer is metastatic cancer.

22. The method of any one of claims 1-21, wherein the subject's colorectal cancer is an adenocarcinoma.

23. The method of any one of claims 1-22, wherein the treatment is administered in the course of one or more 14-day cycles.

24. The method of claim 23, wherein 400 mg of the DKK1 antibody, or antigen binding-fragment thereof is administered on day 1 of the 14-day cycle.

25. The method of claim 23 or 24, wherein the VEGF inhibitor is bevacizumab and is administered at 5 mg/kg on day 1 of the 14-day cycle.

26. The method of any one of claims 23-25, wherein the 14-day cycle is repeated.

27. The method of any one of claims 1-26, wherein the DKK1 antibody, or antigen binding-fragment thereof, comprises a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3, wherein LCDR1 has the amino sequence of SEQ ID NO: 1, LCDR2 has the amino sequence of SEQ ID NO: 2, LCDR3 has the amino sequence of SEQ ID NO:3, HCDR1 has the amino sequence of SEQ ID NO:4, HCDR2 has the amino sequence of SEQ ID NO:5, and an HCDR3 has the amino sequence of SEQ ID NO:6.

28. The method of claim 27, wherein the LCVR comprises the amino acid sequence of SEQ ID NO: 7 and the HCVR comprises the amino acid sequence of SEQ ID NO: 8.

29. The method of claim 27 or 28, wherein the LCVR and HCVR comprise amino acid sequences selected from the group consisting of: (i) a LCVR comprising the amino acid sequence of SEQ ID NO: 9 and a HCVR comprising the amino acid sequence of SEQ ID NO: 10; (ii) a LCVR comprising the amino acid sequence of SEQ ID NO: 11 and a HCVR comprising the amino acid sequence of SEQ ID NO: 12: (iii) a LCVR comprising the amino acid sequence of SEQ ID NO: 13 and a HCVR comprising the amino acid sequence of SEQ ID NO: 10; (iv) a LCVR comprising the amino acid sequence of SEQ ID NO: 14 and a HCVR comprising the amino acid sequence of SEQ ID NO: 10.

30. The method of claim 29, wherein the LCVR comprises the amino acid sequence of SEQ ID NO: 11 and the HCVR comprises the amino acid sequence of SEQ ID NO: 12.

31. The method of claim 30, wherein the DKK1 antibody comprises a heavy chain and a light chain amino acid sequence selected from the group consisting of a) a heavy chain comprising the amino acid sequence of SEQ ID NO: 19 and light chain comprising the amino acid sequence of SEQ ID NO: 16, b) a heavy chain comprising the amino acid sequence of SEQ ID NO: 17 and a light chain comprising the amino acid sequence of SEQ ID NO: 18, c) a heavy chain comprising the amino acid sequence of SEQ ID NO: 19 and a light chain comprising the amino acid sequence of SEQ ID NO: 20, and d) a heavy chain comprising the amino acid sequence of SEQ ID NO: 19 and a light chain comprising the amino acid sequence of SEQ ID NO: 21.

32. The method of claim 31, wherein the DKK1 antibody comprises a heavy chain comprising the amino acid sequence of SEQ ID NO: 17 and a light chain comprising the amino acid sequence of SEQ ID NO: 18.

33. The method of any one of claims 1-32, wherein the DKK1 antibody is DKN-01.

34. The method of any one of claims 1-33, wherein the subject is a human.

35. The method of any one of claims 1-34, wherein the subject is a rapid progressor.

36. The method of any one of claims 1-35, wherein the subject harbors a mutation in a kRas gene or an nRas gene.

37. The method of any one of claims 1-36, wherein the subject suffers from liver metastases.

38. The method of any one of claims 1-37, wherein the colorectal cancer is a rectal cancer.

39. A pharmaceutical composition comprising:
a) a DKK1 antibody, or antigen binding-fragment thereof;
b) a VEGF or a VEGFR inhibitor; and
c) one or more chemotherapeutic agents or a pharmaceutically acceptable salt of any of the foregoing.

40. A pharmaceutical composition comprising:
a) a DKK1 antibody, or antigen binding-fragment thereof;
b) bevacizumab; and
c) one or more chemotherapeutic agents or a pharmaceutically acceptable salt of any of the foregoing.

41. A kit comprising:
a) a DKK1 antibody, or antigen binding-fragment thereof;
b) a VEGF or a VEGFR inhibitor;
c) one or more chemotherapeutic agents or a pharmaceutically acceptable salt thereof; and
d) instructions for use.

42. A kit comprising:
a) a DKK1 antibody, or antigen binding-fragment thereof;
b) bevacizumab;
c) one or more chemotherapeutic agents or a pharmaceutically acceptable salt thereof; and
d) instructions for use.
